(19) United States
(12) Patent Application Publication
(10) Pub. No.: US 2025/0160840 A1
(43) Pub. Date: May 22, 2025

(54) DEVICES FOR IN SITU FORMED, REINFORCED NERVE COAPTATION AIDS, CAPS, AND WRAPS

(71) Applicant: TULAVI THERAPEUTICS, INC., Los Gatos, CA (US)

(72) Inventors: Corinne Bright, Los Gatos, CA (US); Yong Ren, Los Gatos, CA (US); Sophia Michela Campbell, Los Gatos, CA (US)

(21) Appl. No.: 18/954,162

(22) Filed: Nov. 20, 2024

Related U.S. Application Data
(60) Provisional application No. 63/601,527, filed on Nov. 21, 2023.

(51) Int. Cl. A61B 17/11; A61L 27/16; A61L 27/20; A61L 27/52; A61L 27/56

(52) U.S. Cl. CPC A61B 17/1128; A61L 2430/32

(57) ABSTRACT

A method may include positioning a scaffold in at least a portion of a cavity defined by a form. A method may include positioning a first nerve end of a first nerve at least partially on the scaffold in the cavity. A method may include positioning a second nerve end of a second nerve at least partially on the scaffold in the cavity. A method may include introducing an in situ forming media in contact with the first nerve end, the second nerve end, and the scaffold. A method may include permitting the in situ forming media to undergo a transformation from a first, relatively flowable state to a second, relatively non-flowable state to form a protective barrier surrounding the first nerve end and the second nerve end.

### CROSS-REFERENCE TO RELATED APPLICATIONS

This application claims priority to U.S. provisional application 63/601,527 filed on Nov. 21, 2023, which is herein incorporated by reference in its entirety.

### INCORPORATION BY REFERENCE

All publications and patent applications mentioned in this specification are herein incorporated by reference in their entirety, as if each individual publication or patent application was specifically and individually indicated to be incorporated by reference in its entirety.

### TECHNICAL FIELD

This disclosure relates generally to the field of nerve treatments, and more specifically to the field of coaptation aids, caps, and wraps.

### BACKGROUND

Peripheral Nerve Injury (PNI) is a major clinical and public health problem that often leads to significant functional impairment, disability, and chronic pain. While peripheral nerves do have the capacity to regenerate, surgical intervention is often used to repair PNIs. Despite advanced microsurgical techniques, effectively addressing PNIs presents a significant challenge. Outcomes of failed PNI recovery may include muscle atrophy, chronic pain, and weakness. Annually, over 5 million new cases of PNI are anticipated to occur worldwide.

PNIs that necessitate surgical repair can include traumas to the nerve that require resection of a damaged region, resection of neuromas in continuity, or nerve transections due to trauma or surgical damage. The current “gold standard” for repairing transected peripheral nerves is epineural neurorrhaphy, which involves directly suturing the nerve ends together through the epineurium. While suture coaptation has long been the standard of care for primary nerve repair and nerve reconstruction, technical difficulties inherent to the procedure pose significant challenges. These issues include ensuring a tension-free repair; fascicular misalignment due to an overtightened suture coaptation; sutures placed too deeply, causing additional axonal injury; and reactive scarring to foreign material at the anastomosis may all negatively impact axonal regeneration. Importantly, studies have shown the use of sutures in direct nerve coaptation has been correlated with increased scar tissue and decreased nerve conductance. Histopathologic examination of nerves that have been microsurgically repaired using suture show degenerative nerve bundles and prominent granulomas.

Neuromas are benign tumors that arise from neural tissue and are composed of abnormally sprouting axons, Schwann cells, and connective tissue. Even though neuromas can appear following various types of injuries, some of the most common and challenging to treat are derived from trauma or surgical procedures in which neural tissue was damaged or transected. Amputation surgeries necessitate the transection of one or more sensory or mixed nerves. Chronic neuropathic pain, attributed to neuroma formation, develops in up to 30% of patient's post-surgery and results in downstream challenges with wearing a prosthesis. In addition to traumatic and amputation related neuromas, neuromas form across multiple clinical indications such as in general surgery (hernia repair, mastectomy, laparoscopic cholecystectomy), gynecologic surgery (C-section, hysterectomy), and orthopedics (arthroscopy, amputation, knee replacement).

Neuromas develop as a part of a normal reparative process following peripheral nerve injury. They are formed when nerve recovery towards the distal nerve end or target organ fails and nerve fibers improperly and irregularly regenerate into the surrounding scar tissue. Neuromas include a deranged architecture of tangled axons, Schwann cells, endoneurial cells, and perineurial cells in a dense collagenous matrix with surrounding fibroblasts. The upregulation of certain channels and receptors during neuroma development can also cause abnormal sensitivity and spontaneous activity of injured axons. Haphazardly arranged nerve fibers are known to produce abnormal activity that stimulates central neurons. This ongoing abnormal activity can be enhanced by mechanical stimulation, for example, from the constantly rebuilding scar at the injury site.

Neuromas of the nerve stump or neuromas-in-continuity are unavoidable consequences of nerve injury when the nerve is not, or cannot be, repaired and can result in debilitating pain. It has been estimated that approximately 30% of neuromas become painful and problematic. This is particularly likely if the neuroma is present at or near the skin surface as physical stimulation induces signaling in the nerve resulting in a sensation of pain.

The number of amputees in the world has risen significantly in recent years, with war injuries and dysvascular diseases such as diabetes accounting for approximately 90% of all amputee cases. There are currently about 1.7 million amputees living in the United States alone, and over 230,000 new amputee patients are discharged annually from hospitals. Further, it has been estimated that there will be a 20% increase in the number of new amputee cases per year by 2050.

Unfortunately, due to persistent pain in limb remnants, about 25% of amputees are not able to commence rehabilitation, much less resume ordinary daily activities. The cause of such pain can be a neuroma. One recent study reported that 78% of amputees experienced mild to severe pain as a consequence of neuroma formation over the 25-year study period, of which 63% described the pain as constant aching pain. The pain is also frequently described as sharp, shooting, or electrical-like phantom sensations that persist for years after surgical amputation. In addition, patients experience tenderness to palpation of the skin overlying the neuroma, spontaneous burning pain, allodynia, and hyperalgesia.

### SUMMARY

In one aspect, a method of facilitating nerve growth is provided. In some embodiments the method includes, for example, positioning a scaffold in at least a portion of a cavity defined by a form; positioning a first nerve end of a first nerve at least partially onto the scaffold in the cavity; introducing an in situ forming media in contact with the first nerve end and the scaffold; and permitting the in situ forming media to undergo a transformation from a first, relatively flowable state to a second, relatively non-flowable state to form a protective barrier surrounding the first nerve end.

In some embodiments, the method further includes positioning a second nerve end of a second nerve at least partially onto the scaffold in the cavity. In some embodiments, the in situ forming media is further in contact with the second nerve end. In some embodiments, the in situ forming media further forms a protective barrier surrounding the first nerve end and the second nerve end.

In some embodiments, the method further includes positioning a connection target at least partially onto the scaffold in the cavity. In some embodiments, the in situ forming media is further in contact with the connection target. In some embodiments, the in situ forming media further forms a protective barrier surrounding the first nerve end and the connection target.

In some embodiments, the connection target is a prosthesis attachment point or a portion of non-nervous tissue. In some embodiments, the introducing an in situ forming media includes introducing a growth permissive gel and a non-growth permissive gel. In some embodiments, the growth permissive gel is introduced into a gap between the first nerve end and the second nerve end. In some embodiments, the gap is about 0 mm to about 20 mm in length between the first nerve end and the second nerve end. In some embodiments, the gap is about 0 mm to about 10 mm in length between the first nerve end and the second nerve end. In some embodiments, the growth permissive gel does not integrate into the scaffold. In some embodiments, the non-growth permissive gel integrates into the scaffold. In some embodiments, the growth permissive gel includes hyaluronic acid and water.

In some embodiments, the growth permissive gel further includes chitosan, polylysine, collagen, fibronectin, poly-L-ornithine, and laminin. In some embodiments, the growth permissive gel is about 0.01 wt % to about 0.1 wt % polylysine, about 3 wt % to about 6 wt % collagen, and about 0 wt % to about 0.5 wt % laminin. In some embodiments, the non-growth permissive gel includes polyethylene glycol and water. In some embodiments, the non-growth permissive gel is about 3 wt % to about 15 wt % solid content. In some embodiments, the introducing the in situ forming media provides a coverage length of about 3 mm to about 20 mm on the first nerve end and the second nerve end.

In some embodiments, the introducing the in situ forming media provides a coverage length of about 3 mm to about 10 mm on the first nerve end and the second nerve end. In some embodiments, the introducing the in situ forming media provides a coverage length of about 5 mm to about 8 mm on the first nerve end the second nerve end. In some embodiments, a thickness of the scaffold is less than or equal to about 1 mm. In some embodiments, a thickness of the scaffold is about 0.8 mm to about 1.2 mm. In some embodiments, a thickness of the scaffold is about 1 mm. In some embodiments, the scaffold has a plurality of pores. In some embodiments, the plurality of pores of the scaffold are each about 0.8 mm2 to about 1.2 mm2 in cross-sectional area.

In some embodiments, the scaffold is biodegradable. In some embodiments, the scaffold biodegrades in about 1 month to about 6 months. In some embodiments, the scaffold biodegrades in about 6 weeks to about 8 weeks. In some embodiments, the scaffold includes or is formed of a technical textile. In some embodiments, the scaffold includes or is formed of a knitted material. In some embodiments, the scaffold includes or is formed of a warp knitted material. In some embodiments, the scaffold includes or is formed of a woven material. The method of claim 1, wherein the scaffold includes or is formed of a natural monofilament, polydioxanone, poly(lactic-co-glycolic acid), poliglecaprone, polyglactin, polyglycolic acid, polyglycolide fiber, polylactide fiber, collagen, alginate, chitosan, cellulose, or carboxymethylcellulose. In some embodiments, the scaffold includes or is formed of polyglycolide fiber. In some embodiments, the scaffold includes or is formed of warp knitted polyglycolide fiber. In some embodiments, the scaffold is hydrophilic. In some embodiments, the first nerve end is positioned in contact with the scaffold within the cavity.

In some embodiments, the method further includes offsetting, using the scaffold, the first nerve end from a sidewall of the form. In some embodiments, the scaffold is configured to produce friction on the first nerve end effective to hold the first nerve end in a substantially static position during the introducing of the in situ forming media. In some embodiments, the first nerve end and the second nerve end are positioned in contact with the scaffold within the cavity.

In some embodiments, the method further includes offsetting, using the scaffold, the first nerve end and the second nerve end from a sidewall of the form. In some embodiments, the scaffold is configured to produce friction on the first nerve end and the second nerve end effective to hold the first nerve end and the second nerve end in a static position during the introducing of the in situ forming media. In some embodiments, the scaffold has a static coefficient of friction of about 0.8 to 1. In some embodiments, the scaffold has an elastic modulus of at least about 1 kPa. In some embodiments, the scaffold has an clastic modulus of about 15 kPa to about 45 kPa. In some embodiments, the scaffold has a tensile strength of at least about 10 N. In some embodiments, the scaffold has a tensile strength of about 70 N to about 100 N. In some embodiments, the in situ forming media is biodegradable. In some embodiments, the in situ forming media biodegrades in about 3 months to about 3 years. In some embodiments, the in situ forming media biodegrades in about 3 months to about 6 months. In some embodiments, the method further includes removing the form.

In another aspect, a system for creating a protective barrier in situ around a nerve end or nerve to nerve junction is provided. In some embodiment, the system includes, for example, a form including a sidewall defining a cavity and a top opening for accessing the cavity, the top opening configured to receive a first nerve end; and a scaffold at least partially positioned on a bottom interior surface of the sidewall of the form such that the first nerve end is offset from the bottom interior surface of the sidewall when the first nerve end is positioned on the scaffold within the cavity.

In some embodiments, the top opening is configured to receive a second nerve end. In some embodiments, the second nerve end is offset from the bottom interior surface of the sidewall when the second nerve end is positioned on the scaffold within the cavity. In some embodiments, the top opening is configured to receive a connection target. In some embodiments, the connection target is offset from the bottom interior surface of the sidewall when the connection target is positioned on the scaffold within the cavity. In some embodiments, the connection target is a prosthesis attachment point or a portion of non-nervous tissue. In some embodiments, the top opening has a first cross-sectional area being less than a second cross-sectional area of the cavity. In some embodiments, the form further includes a first nerve guide including a first surface at least partially defining a first side access through the sidewall for positioning a first nerve end in the cavity, the first surface being on a first end of the form at a first elevated position relative to a bottom interior surface of the sidewall.

In some embodiments, the system further includes a second nerve guide including a second surface at least partially defining a second side access through the sidewall for positioning a second nerve end in the cavity, the second surface being on a second end of the form at a second elevated position relative to a bottom interior surface of the sidewall. In some embodiments, the system further includes an in situ forming media configured to be introduced into the form and to form a protective barrier at least partially around a circumference of the first nerve end. In some embodiments, the system further includes an in situ forming media configured to be introduced into the form and to form a protective barrier at least partially around a circumference of the first nerve end and second nerve end. In some embodiments, the in situ forming media includes a growth permissive gel configured to be introduced into a gap between the first nerve end and the second nerve end when the first nerve end and the second nerve end are positioned on the scaffold within the cavity of the form. In some embodiments, the in situ forming media includes a non-growth permissive gel configured to at least partially integrate into the scaffold.

In some embodiments, the in situ forming media is biodegradable. In some embodiments, the in situ forming media biodegrades in about 3 months to about 3 years. In some embodiments, the in situ forming media biodegrades in about 3 months to about 6 months. In some embodiments, the scaffold includes or is formed of a technical textile. In some embodiments, the scaffold includes or is formed of a knitted material. In some embodiments, the scaffold includes or is formed of a warp knitted material. In some embodiments, the scaffold includes or is formed of a woven material. In some embodiments, the scaffold includes or is formed of a natural monofilament, polydioxanone, poly(lactic-co-glycolic acid), poliglecaprone, polyglactin, polyglycolic acid, polyglycolide fiber, polylactide fiber, collagen, alginate, chitosan, cellulose, or carboxymethylcellulose. In some embodiments, the scaffold includes or is formed of polyglycolide fiber. In some embodiments, the scaffold includes or is formed of warp knitted polyglycolide fiber. In some embodiments, the scaffold is hydrophilic. In some embodiments, the scaffold has a thickness of less than or equal to about 1 mm. In some embodiments, the scaffold has a thickness of about 0.8 mm to about 1.2 mm.

In some embodiments, the scaffold has a thickness of about 1 mm. In some embodiments, the scaffold has a coefficient of friction of about 0.8 to 1. In some embodiments, the scaffold has an elastic modulus of at least about 1 kPa. In some embodiments, the scaffold has an elastic modulus of about 15 kPa to about 45 kPa. In some embodiments, the scaffold has a tensile strength of at least about 10 N. In some embodiments, the scaffold has a tensile strength of about 70 N to about 100 N. In some embodiments, the scaffold has a plurality of pores. In some embodiments, the plurality of pores of the scaffold are each about 0.8 mm2 to about 1.2 mm2 in cross-sectional area. In some embodiments, the scaffold is biodegradable. In some embodiments, the scaffold biodegrades in about 1 month to about 6 months. In some embodiments, the scaffold biodegrades in about 6 weeks to about 8 weeks.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1A shows an embodiment of a form.

FIG. 1B shows a cross-section of an embodiment of a form

FIG. 2 shows an embodiment of a scaffold positioned in in the form with partial transparency.

FIG. 3A shows an embodiment of a first nerve end positioned in a form with a scaffold.

FIG. 3B shows an embodiment of a first nerve end and a second nerve end positioned in a form with a scaffold.

FIG. 3C shows an embodiment of a first nerve end and a connection target positioned in a form with a scaffold.

FIG. 3D shows an embodiment of a first nerve end and a second nerve end positioned in a form with a scaffold and a growth permissive gel as in situ forming media introduced in between the nerve ends.

FIG. 4A shows an embodiment of a form, scaffold, and first and second nerve ends after introduction of a non-growth permissive gel as in situ forming media.

FIG. 4B shows a cross-section of an embodiment of a form, scaffold, and a nerve end after introduction of a non-growth permissive gel as in situ forming media.

FIG. 5A shows a perspective view of an embodiment of a nerve coaptation site connected with an embodiment of a scaffold in in situ forming media.

FIG. 5B shows a side view of an embodiment of a nerve coaptation site connected with an embodiment of a scaffold in in situ forming media.

FIG. 6A shows an example of weft knitting.

FIG. 6B shows an example of warp knitting.

FIG. 7 shows a graph comparing break force for a bimorphic polymer, sutures, hydrogel in a form, and hydrogel in a form with a scaffold.

FIG. 8 depicts a flowchart for a method of facilitating nerve growth.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the claimed subject matter. Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

In treating PNIs, minimizing the use of suture at the coaptation site could potentially improve nerve functional outcomes after PNI by minimizing iatrogenic injury and complications. Further, epineural neurorrhaphy requires a high level of surgical precision due to the delicate nature of peripheral nerves. Surgeons must handle the nerve tissue with extreme care to avoid further damage, resulting in a lengthy procedure and dependence on microsurgical techniques. The use of coaptation aids to mitigate the inherent challenges of peripheral nerve repair may provide an alternative to suture repair. These offer advantages over traditional suture-based techniques that include facilitating a tension-free repair, reducing operative time, and potentially improving functional outcomes.

Coaptation aids largely feature dehydrated polymeric or animal-derived extracellular matrix (ECM) materials that wrap around a coaptation site in a tube formation. Examples such as Integra® NeuraGen® Nerve Guides, Axogen® Nerve Guards, Polyganic NEUROLAC®, and Biocircuit Technologies® Nerve Tape®, come as preformed tubes in various sizes or are created by the surgeon in the operative room by wrapping of a sheet of these materials around the nerve junction. However, these coaptation aids fail to fully conform to the nerve ends' shape, and these aids all use an attachment method that pierces the nerve epineurium such as sutures or nitinol hooks.

There exists a need for a device that aids in the management of peripheral nerve discontinuities and provides a protected environment for nerve regeneration, while at the same time reducing surgical duration and complexity. The devices and methods described herein address these needs and aid in suture-less transected peripheral nerve repair without piercing the nerve epineurium and fully conforming to the nerves' shape and size. The devices and methods described herein protect PNIs by serving as a barrier between the nerve and the surrounding tissue, reducing or eliminating the need for microsurgical techniques, and easing the technical difficulty of the peripheral nerve repair surgery.

While various methods have been used to prevent, minimize, or shield neuromas in an attempt to minimize neuropathic pain, the current clinical “gold standard” for treating neuromas is traction neurectomy, in which the nerve is pulled forward under traction and transected as far back as possible in the hope that, if a neuroma forms, that it will be located deep in the tissue. Another well recognized approach is to bury the proximal nerve end (that will form the neuroma) into muscle or a hole drilled in bone. The nerve is then sutured to the muscle or periosteum of the bone to maintain its position. The rationale for this is that the surrounding tissue cushions and isolates the neuroma to inhibit stimulation and the resulting painful sensations. However, this procedure can greatly complicate surgery, as significant additional dissection of otherwise healthy tissue is required to place the nerve stump. This, coupled with poor and variable efficacy, the lack of appropriate/available tissue, and the additional procedural time required, result in the procedure being rarely performed to prevent neuroma formation.

Another method is to cut the nerve stump back to leave a segment or sleeve of overhanging epineurium. This overhang can be ligated to cover the face of the nerve stump. Alternatively, a segment of epineurium can be acquired from other nerve tissue or a corresponding nerve stump can be cut back to create an epineurium sleeve that can be used to connect with and cover the other nerve stump.

Yet another method that is commonly used is a suture ligation, where a loop of suture is placed around the end of the nerve and tightened. This pressure is believed to mechanically block the exit of axons and causes the terminal end to eventually form scar tissue over the site. Clinical and pre-clinical evidence has shown, however, that this procedure can cause a painful neuroma to form behind the ligation. Furthermore, the ligated nerve is generally not positioned to minimize mechanical stimulation of the neuroma, since it is anticipated that the scar tissue will provide sufficient protection to the nerve end.

Other methods used clinically include placing the nerve stump within a solid implantable silicone or biodegradable polymer tube with an open, or more recently, a sealed end (e.g. Polyganics NEUROCAP®); or wrapping the proximal nerve end with a harvested vein or fat graft, with the goal of providing a physical barrier to aberrant nerve regeneration. The use of biomaterial implant devices and methods necessitate insertion and securing the nerve with sutures in the opening of the device, which can be difficult and further damage the nerve end. For example, the current procedure for securing the NEUROCAP® uses a suture placed in the epineurium of the nerve and through the wall of the tube and followed by pulling and stuffing the nerve into the lumen of the tube using the suture and the placement of several sutures to retain the nerve in the device. These methods and devices can also result in mechanical stimulation of the neuroma tissue as a result of 1) mismatch between the tissue compliance and the rigidity of the conduit; and 2) inability of the cap to prevent neuroma formation within the cap, with resulting sensation of pain. Although these nerve caps degrade over a period of 3 months to 18 months, substantial degradation-mediated mass loss occurs in the first three to six months resulting in the exposure of a temporarily protected neuroma to the surrounding environment. Thus, the efficacy of these solid implantable caps is limited by the ability of the cap to conform to the proximal end of the nerve and prevent neuroma formation and secondarily their subsequent degradation to expose the neuroma to the surrounding environment. Finally, since these methods require suturing using fine sutures (9-0 nylon), the procedural time and skill needed to secure these implants under surgical magnification (loupes) or harvested tissue prohibits surgeons from more broadly adopting these procedures.

Unfortunately, current methods for addressing the formation of and pain caused by neuromas have not been widely adopted. The need therefore remains for an effective technology or therapy for controlling or inhibiting neuroma formation following inadvertent or planned surgical or traumatic nerve injury in addition to reducing scar formation and perineural adhesions.

A variety of biomaterial conduits have been explored preclinically to try to prevent neuroma formation, including other solid implantable biodegradable polymeric conduits based on polylactide/polycaprolactone, atelocollagen, porcine small intestine submucosa, or microcrystalline chitosan. These approaches have not been successful to date in preventing the formation of neuromas either because the solid implants do not form in situ and create a potential space to permit nerve outgrowth and varying degrees of neuroma formation or because the in vivo persistence of the materials was not sufficient to prevent neuroma formation.

The disclosure herein presents technical solutions for at least some of the technical problems described above. The in situ forming implants described herein can be compliant with the surrounding tissue, encompass but do not compress the underlying nerve tissue, and are flexible such that they can move over regions of tissue involving joints or where nerves slide relative to other tissues. These material features obviate or reduce the surgical expertise, collateral tissue damage, and expense of suturing, dragging or stuffing of the nerve inside a conduit as used in existing techniques. By conforming to the end(s) of the nerve(s) without compression, the implants and methods described herein also provide a physical barrier to nerve regeneration while avoiding the mechanical stressors now evidenced to cause painful scar tissue and neuroma formation around the nerve. Additionally, the in situ forming implants provide mechanical strength to support nerve regeneration for a period of two months, three months, or more to prevent axonal escape during the growth regenerative phase after nerve injury. This period of strength paired with the implants' capacity for a timed degradation and absorption into a subject's tissues is a noted advantage over pre-existing solid implants.

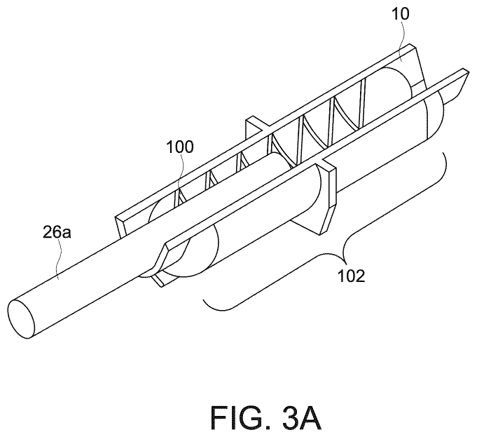

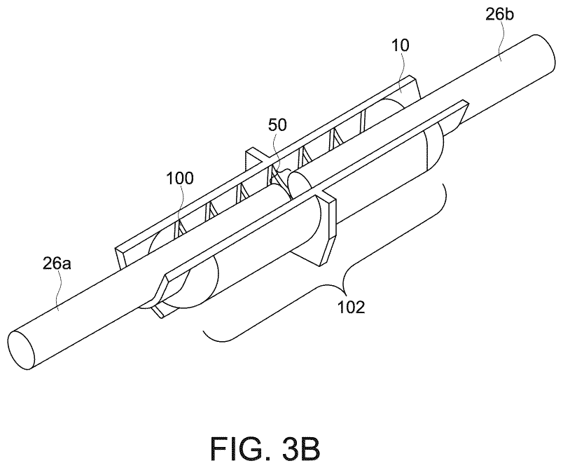

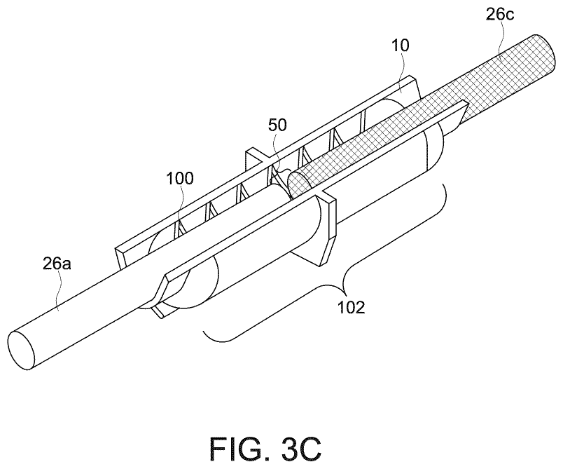

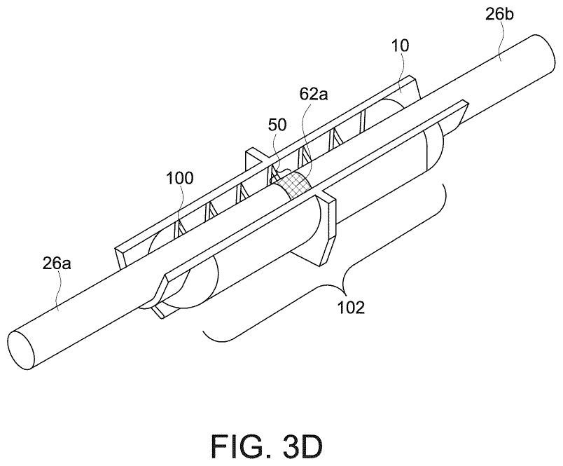

The technology described herein may be used to prevent nerve outgrowth into the surrounding tissue and direct the outgrowth of a transected or compressed nerve into the distal nerve stump or allograft/autograft. In some aspects, the suture-free technology as described herein serves as a technological solution that can direct nerve regeneration from a proximal nerve stump directly (through direct coaptation/anastomoses with a distal nerve stump), indirectly (through a nerve conduit, guidance channel, allograft, autograft), or through a growth-permissive matrix (e.g., growth permissive gel 62a of FIG. 3D) into the distal nerve stump. In addition, in some aspects, the technology described herein allows for a de-tensioning of anastomoses sites that promote better nerve regeneration over established techniques incapable of providing adequate de-tension. Lastly, in some aspects, the implants described herein are sufficiently simple in implantation to be quickly and broadly applied to nerves to prevent inadvertent damage to adjacent nerves more easily than other preexisting surgical techniques and implants.

In general, the devices and methods described herein may be used in situ for connecting and repairing nerves, for example, nerves that were cut as a result of trauma or transected as a result of a surgical procedure. In some embodiments, the devices and methods may be used to repair nerves that can be repaired directly with minimal, negligible, or no tension in which the proximal and distal nerve ends can be brought together, referred to a direct coaptation or direct repair. In some embodiments, gaps of between about 0 mm to about 5 mm permit direct coaptation repair. In some embodiments, the devices and methods are used to repair small or medium gaps, up to an about 10 mm gap between the two nerve ends in which direct repair is not possible. In some embodiments, the devices and methods are used to repair large gaps, including gaps between about 10 mm to about 20 mm or longer, including about 25 mm to about 35 mm gaps or longer gaps. The devices and methods described herein permit the alignment and/or guidance of nerves and the prevention of fascicular escape, to facilitate an increased number of axons generating towards their distal target and thus an increased number of nerves with functionally relevant reinnervation of the distal targets. The devices and methods permit suture-less nerve repair, or the repair of nerves without the use of sutures.

In some embodiments, the devices and methods are used to connect a nerve with autologous or allograft nerve tissue and provide coaptation repair between the two tissues, either at the first coaptation site (proximal nerve stump to allograft/autograft nerve) or at the second distal coaptation site (allograft/autograft) to distal nerve stump, or both. In other embodiments, the device can be adapted for use with other biomaterials used for coaptation, including tubes, tubes with slits, and sheets, that are wrapped around nerves.

In some embodiments, the devices and methods can be incorporated into other gels, such as gels that form around the end of a nerve to provide a nerve cap or gels that are delivered around an intact nerve to provide a nerve wrap or protective layer around a nerve. In some embodiments, the devices and methods described herein may be used in situ to prevent neuroma formation. Some aspects of the present disclosure involve in situ formation of a protective barrier around a nerve end or two nerve ends using injectable or surgically introduced media which may be a gel, hydrogel, or gel precursors to block nerve regeneration and/or neuroma formation, inflammation, and/or adhesion, and the like around and/or in contact with nerves. In some embodiments, in situ formation of a protective barrier may be used to bond or couple two nerve ends together until they bond together without assistance or intervention. Access may be by way of an open surgical approach or endoscopic, such as through a port. The nerve end or stump may be formed by transection (cutting), traumatic injury, or ablation through any of a variety of modalities including radiofrequency, cryo, ultrasound, chemical, thermal, microwave, or other methods known in the art.

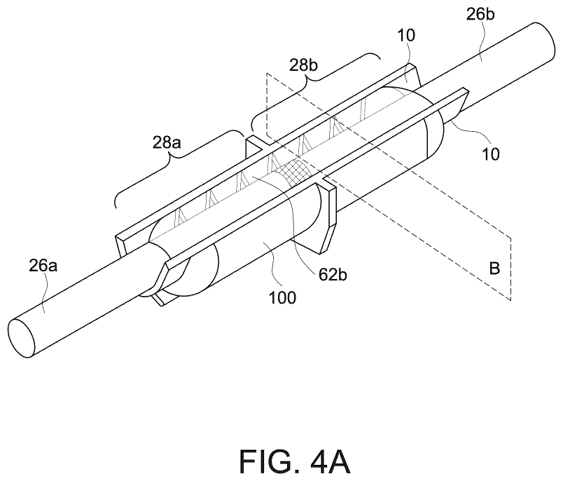

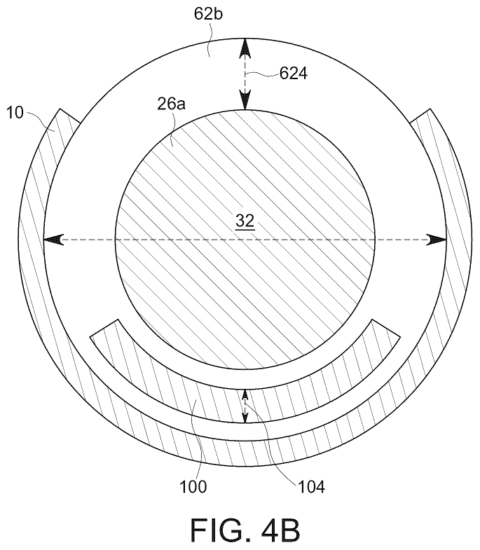

In various embodiments, a scaffold having material properties as described herein provides technical solutions to overcome a number of technical challenges for the above-described techniques. For example, the scaffold as described herein aids in the positioning and alignment of nerve ends without sagging or drooping, and better secures them in the desired positions during the introduction of an in situ forming media (e.g., a hydrogel, growth permissive gel, nongrowth permissive gel, etc.). Additionally, in some embodiments, the material properties of the scaffold permit both the restriction of an optional first component of an in situ forming media (e.g., a growth permissive gel 62a of FIG. 3D) to a desired location between the nerve ends and the free distribution of a second component of the media (e.g., a non-growth permissive gel 62b of FIG. 4A) around the nerve ends without interfering or impeding the cross-linking of the in situ forming media. Furthermore, the scaffold provides additional mechanical strength to the coaptation site during the recovery process without the use of compression or substantial tension on the nerve ends.

At a high level, the system, devices, and methods herein pertain, in various embodiments, to a form, with or without a removable scaffold, for positioning within a first nerve end alone or in proximity to a second nerve end or connection target (e.g., a prosthesis attachment point or a portion of non-nervous tissue, etc.) An in situ forming media (e.g., a hydrogel) may be introduced into the form, at least partially covering the one or more nerve ends and scaffold. Once the in situ forming media has transformed from a first, relatively flowable state to a second, relatively non-flowable state to form a protective barrier surrounding the nerve ends (i.e., at least partially around a circumference of the nerve ends), the form may be removed. In at least this way, the system, devices, and methods described herein allow for the creation of a protective barrier in situ around a nerve end, nerve to nerve junction, or nerve to target junction.

### Form and Scaffold

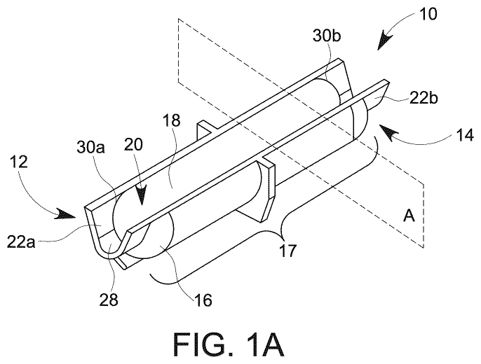

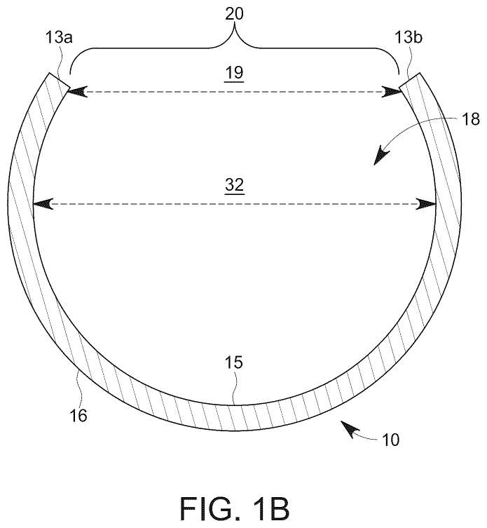

FIG. 1A shows a form 10. The form 10 extends between a first end 12 and a second end 14 and includes a side wall 16 extending between the first end 12 and the second end 14. Sidewall 16 is at least partially concave or nonplanar to define a cavity 18. The form cavity 18 is at least partially exposed to an outside of the form 10 by way of a top opening 20. In some embodiments, the top opening 20 may receive one or more nerves or connection targets (e.g., a first nerve end 26a of FIG. 3A, a first and second nerve end 26a and 26b of FIG. 3B, a first nerve end 26a and connection target 26c of FIG. 3C). In various embodiments, the sidewall 16 may extend anywhere between about 120 degrees (e.g., partially circumferential) to substantially 360 degrees (fully circumferential), including at about 180 degrees (e.g., semi-circular). In some embodiments, the sidewall 16 may extend about 120 degrees to about 320 degrees, about 120 degrees to about 240 degrees, about 120 degrees to about 180 degrees, about 120 degrees to about 160 degrees, about 160 degrees to about 320 degrees, about 160 degrees to about 240 degrees, about 180 degrees to about 320 degrees, about 180 degrees to about 320 degrees, or about 180 degrees to about 240 degrees.

One or both of the first end 12 and the second end 14 of the form 10 may include a nerve guide (e.g., a first and second nerve guide 22a and 22b, respectively) to facilitate passage of a nerve (e.g., nerves ends 26a and 26b of FIG. 3B), in some embodiments, to position a nerve end or connection target within the cavity 18. The nerve guide 22 includes an upwardly concave support surface 28 located exterior to a nerve entrance portal 30a and 30b into the cavity 18 of the form 10. The entrance portals 30a and 30b define, in some embodiments, a first and second side access through the sidewall 16 into the cavity 18. The support surface 22 supports the nerve end within the form cavity 18 at an elevated position relative to a bottom interior surface 15 (see FIG. 1B) of the sidewall 16.

In some embodiments, the nerve entrance portals 30a and 30b are identically sized, such as for nerve repair with two nerves of a similar size. In some examples, the nerve entrance portals 30a and 30b may vary to accommodate different nerve sizes such as for a mismatch in the diameter of a proximal nerve stump and a distal nerve stump that are to be repaired. For example, a first nerve entrance portal 30a may be a larger size than a second nerve entrance portal 30b, to accommodate a difference in nerve sizes between the proximal and the distal nerve stump, as may occur in a nerve transfer procedure. In other embodiments, a surgeon performing the procedure trims the form 10 to the size needed or punches at least one nerve entrance portal 30a or 30b into the form 10 to the size needed instead of the form 10 being manufactured with preexisting nerve entrance portals 30a and 30b of a predefined size.

FIG. 1B shows a cross-section of the embodiment of FIG. 1A along plane A. In some embodiments, an internal diameter 32 of the cavity 18 of the form 10 is greater than the distance 19 across top opening 20 between a first top edge 13a and an opposing second top edge 13b of the sidewall 16. When the distance 19 and the internal diameter 32 are each multiplied by the length 17 (see FIG. 1A) of the form 10, the top opening 20 has a first cross-sectional area that is less than a second cross-sectional area of the cavity 18, as shown in FIG. 1B. In some embodiments, when the sidewall 16 defines a semi-circular cavity 18, the distance 19 across top opening 20 between the first top edge 13a and the opposing second top edge 13b is equal to the internal diameter 32 of the cavity 18 of the form 10. In these embodiments, the top opening 20 has a first cross-sectional area that is equal to a second cross-sectional area of the cavity 18.

The nerve entrance portals 30a and 30b and the internal diameter 32 of the cavity 18 of the form 10 may accommodate nerves ranging in size from about 0.1 mm in diameter to about 20 mm in diameter, including mismatches nerve sizes, e.g., an about 8 mm diameter nerve connected to an about 1 mm diameter nerve, or an about 4 mm diameter nerve connected to an about 2 mm diameter nerve. The length 17 of the form 10 may be adapted depending on the application and the desired length of coverage (e.g., coverage lengths 28a and 28b of FIG. 4A) of the in situ forming media (e.g., one or both of the growth permissive gel 62a of FIG. 3D and non-growth permissive gel 62b of FIG. 4A) around the nerve ends 26a and 26b as described herein. In some embodiments, the length 17 of the form 10 may be about 10 mm to about 30 mm, or about 12 mm to about 25 mm.

In some embodiments, the form 10 is made of a nonadherent, nondegradable material, such as, but not limited to, medical-grade silicone, polydimethylsiloxane (PDMS), and various plastics and elastomers, sufficiently flexible to be peeled, popped off, or released from the in situ forming material (e.g., non-growth permissive gel 62b of FIG. 4A with or without growth permissive gel 62a of FIG. 3D). In some embodiments, the form 10 is made of a hydrophobic material. In some embodiments, the form 10 is made of a non-toxic material. In some embodiments, the material of the form 10 has a durometer type A scale value of about 20 to about 40, about 20, about 30, or about 40. In some embodiments, the form 10 may be colored to provide contrast against the surrounding tissue during use so that the form 10 is not accidentally left in situ by a user.

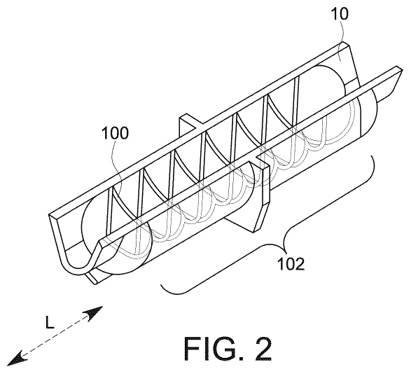

FIG. 2 shows a scaffold 100 having a length 102 positioned in in the form 10, the form 10 depicted with partial transparency to show the scaffold 100. In some embodiments, the scaffold 100 is at least partially positioned on a bottom interior surface 15 of the form 10 (e.g., on a bottom interior surface 15 of the sidewall 16 of the form 10). Said another way, the scaffold 100 may be at least partially positioned on a surface 15 (e.g., a concave surface or nonplanar surface) in a cavity 18 defined by the form 10. In some embodiments, the scaffold 100 provides a higher friction surface which holds or retains the nerve or nerves (e.g., nerve ends 26a and 26b of FIG. 3A) in a desired position during the delivery of the in situ forming media (e.g., non-growth permissive gel 62b of FIG. 4A with or without growth permissive gel 62a of FIG. 3D). In this manner, it may be understood that the scaffold 100 holds or maintains the nerves or nerve ends 26a and 26b under minimal tension. In some embodiments, the minimal tension may be approximately 0 newtons (N) or less than about 1 N, less than about 0.5 N, less than about 0.25 N, between about 0 N to about 1N, between about 0 N to about 0.25 N, between about 0 N to about 0.5 N, between about 0 N to about 0.75 N, etc. Low or minimal tension has been demonstrated to be beneficial to nerve repair. The scaffold 100 may provide mechanical support to the in-situ forming media as well as to permit hands-free coaptation of the nerve or nerve ends 26a and 26b. In some embodiments, the scaffold 100 functions as a spacer such that the scaffold 100 offsets a nerve end (e.g., nerve end 26a or 26b of FIGS. 3A and 3B) from a sidewall 16 of the form 10 and prevents the nerve ends 26a and 26b from drooping or sagging (or reduces drooping) when entering the cavity 18 of the form 10 or positioned in the form 10.

In some embodiments, the scaffold 100 is made of a matrix or is formed of a technical textile (i.e., a textile manufactured for functional properties rather than aesthetics) having a particular pore size, wettability, structural design, flexibility, and/or stretchability as described herein. For example, the scaffold 100 may be made of a natural monofilament (e.g., made from cow, sheep, pig, or goat intestines, amnion, or chorion), polydioxanone, Poly(lactic-co-glycolic acid) (PLGA), poliglecaprone (e.g., glycolide and epsilon-caprolactone copolymer), polyglactin (e.g., braided filaments of glycolide and L-lactide), polyglycolic acid (PLA), polyglycolide fiber (PGA), polylactide fiber, collagen, alginate, chitosan, cellulose, cellulose derivatives including, but not limited to, carboxymethylcellulose, or a combination thereof. In some embodiments, the scaffold 100 may be made of collagen, alginate, or a natural monofilament. In some embodiments, the scaffold 100 is made of a natural monofilament. In some embodiments, the scaffold is made of alginate, chitosan, cellulose, or a cellulose derivative. In some embodiments, the scaffold 100 is made of polydioxanone, poly(lactic-co-glycolic acid) (PLGA), poliglecaprone (e.g., glycolide and epsilon-caprolactone copolymer), polyglactin (e.g., braided filaments of glycolide and L-lactide), polyglycolic acid (PLA), polyglycolide fiber (PGA), polylactide fiber. In some embodiments, the scaffold is made of PLGA, PGA, collagen, or alginate.

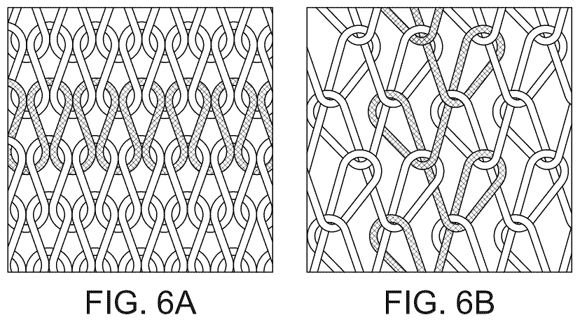

In some embodiments, the material of the scaffold 100 may be knitted material (e.g., weft knitted, warp knitted, etc.) or woven material made of monofilaments or braided filaments. An example of weft knitting is shown in FIG. 6A. Further, an example of warp knitting is shown in FIG. 6B. In weft knitting, a filament runs in a horizontal row and is looped through adjacent rows ahead and behind it. In warp knitting, a filament runs in a vertical line and is looped through adjacent lines to the left and right of it. Generally speaking, weft knitted materials are more stretchable and flexible compared to warp knitted materials, but warp knitted materials are more durable and rigid than weft knitted materials. In some embodiments, the scaffold 100 includes or is at least partially a warp knitted scaffold including or at least partially formed of PGA. In some embodiments, the material of the scaffold 100 may be electrospun. In some embodiments, the material of the scaffold 100 may be casted or molded and lyophilized. A variety of techniques may be employed to manufacture the scaffold 100 without deviating from the scope of this disclosure.

In some embodiments, the scaffold 100 may feature pores of a sufficient size such that at least part of the in situ forming media (e.g., the non-growth permissive gel 62b of FIG. 4A) can flow around the one or more nerve ends 26a and 26b and through the pores of the scaffold 100 to integrate into the scaffold 100 without generating bubbles or suffering a loss in flowability and capacity for polymerization. For example, individual pores of a scaffold 100, in some embodiments, may have a size of about 0.2 mm² to about 2 mm², about 0.5 mm² to about 1.5 mm², or about 0.8 mm² to about 1.2 mm² in cross-sectional area. In various embodiments, pores of about 0.2 mm² to about 2 mm², about 0.5 mm² to about 1.5 mm², or about 0.8 mm² to about 1.2 mm² in cross-sectional area may permit facile penetration and integration of the scaffold 100 by at least part of the in situ forming media (e.g., the non-growth permissive gel 62b of FIG. 4A) while still producing sufficiently strong material properties (e.g., rigidity, elasticity, etc.), as described herein.

In some embodiments, the scaffold 100 may be hydrophilic and/or substantially wettable such that the scaffold 100 does not inhibit the flowability and/or polymerization of at least part of the in situ forming media (e.g., that of the non-growth permissive gel 62b of FIG. 4A) nor cause the generation of bubbles in the in situ forming media during the introduction or delivery of the media into the form 10. In some embodiments, a droplet of water may fully penetrate the scaffold 100 within, on average, about 0.2 seconds to about 3 seconds, about 0.3 seconds to about 3 seconds, about 0.3 seconds to about 2 seconds, about 0.3 seconds to about 1.5 seconds, about 0.3 seconds to about 1 second, about 0.3 seconds to about 0.75 seconds, or less than about 1 second. In at least this manner, some embodiments of the scaffold may be said to be substantially wettable.

In some embodiments, the scaffold 100 is biodegradable (the terms “biodegrade” and “degrade” are used herein synonymously.) For example, the scaffold 100 may degrade in-situ before the in-situ forming media (e.g., a hydrogel) degrades in-situ. In some embodiments, the scaffold degrades in about 1 month to about 6 months, about 3 months to about 6 months, about 1 month to about 3 months, about 1 month to about 2 months, or about 6 weeks to about 8 weeks. For example, a scaffold 100 at least partially including PLA may degrade in about 3 months to about 6 months. For example, a scaffold 100 at least partially including PLGA may degrade in about 1 month to about 6 months. For example, a scaffold 100 at least partially including chitosan may degrade in about 1 month to about 3 months. For example, a scaffold 100 at least partially including alginate may degrade in about 1 month to about 2 months. In some embodiments, the scaffold 100 at least partially including PLGA degrades in situ in about 6 weeks to about 8 weeks.

As shown in FIG. 3A, a first nerve end 26a may be at least partially placed onto the scaffold 100 positioned in a form 10. In embodiments that place only a single nerve end 26a onto the scaffold 100 in the form 10, the devices and methods described herein may serve to cap the nerve end 26a in in situ forming media. In some embodiments, a second nerve end 26b may be at least partially placed onto the scaffold 100 positioned in the form 10 as shown in FIG. 3B. In embodiments that at least partially place a first and a second nerve end 26a and 26b onto the scaffold 100 in the form 10, the devices and methods described herein may serve to form a coaptation site for a nerve-to-nerve repair junction. In some embodiments, such as that depicted in FIG. 3C, the second nerve end 26b may be replaced by a connection target 26c. Example connection targets 26c include, but are not limited to, a prosthesis attachment point, a portion of non-nervous tissue (e.g., muscle, bone, connective tissue, organ tissue, etc.), and the like. In embodiments that at least partially place a first nerve end 26a and a connection target 26c onto the scaffold 100 in the form 10, the devices and methods described herein may serve to form a nerve anchor site. For simplicity of language, many embodiments depicted and described herein may refer to a first and second nerve end 26a and 26b; however, these embodiments are intended to also include embodiments in which the second nerve end 26b is replaced by a connection target 26c, even if not expressly stated.

In some embodiments, the scaffold 100 has a curvature, or is nonplanar, to permit it to cover between about 10% to about 95%, about 10% to about 80%, about 10% to about 75%, about 10% to about 65%, about 10% to about 50%, about 10% to about 35%, about 10% to about 30%, about 25% to about 95%, about 25% to about 80%, about 25% to about 75%, about 25% to about 65%, about 25% to about 50%, about 25% to about 35%, or about 25% to about 30% of the circumference of the nerve ends 26a and 26b when the nerve ends 26a and 26b are appropriately placed within the scaffold 100 and form 10. In some embodiments, the scaffold 100 has a radius of curvature of about 1 mm to about 4 mm, about 1.5 mm to about 4 mm, about 2 mm to about 4 mm, about 2.5 mm to about 4 mm, about 3 mm to about 4 mm, about 3.5 mm to about 4 mm, or about 3.8 mm. In embodiments in which the scaffold 100 has a radius of curvature of about 2 mm to about 4 mm, the scaffold 100 may accommodate a form 10 having up to about 7 mm in internal diameter 32. In some embodiments, scaffold 100 may be pre-curved or pre-formed with a curvature to accommodate the form 10 and the nerve ends 26a and 26b. The scaffold 100 may be cut to smaller sizes with the same radius to accommodate smaller sizes of nerve ends 26a and 26b or smaller sizes of a form 10, in some embodiments.

To support the nerve ends 26a and 26b, in some embodiments, the scaffold 100 is sufficiently rigid such that the nerve ends 26a and 26b do not crush the scaffold 100 or cause the scaffold 100 to collapse. In some embodiments, the scaffold 100 has an elastic modulus of at least about 1 kPa. In some embodiments, the scaffold 100 has an elastic modulus of about 10 kPa to about 50 kPa, about 15 kPa to about 45 kPa, about 20 kPa to about 40 kPa, about 25 kPa to about 35 kPa, or about 30 kPa. In at least this manner, the scaffold 100 functions to reduce sagging or drooping of the first and second nerve ends 26a and 26b in the form 10. In some embodiments, the scaffold 100 may function to align the nerve ends 26a and 26b positioned in the form 10, such that the nerve ends 26a and 26b rest on the scaffold 100. Said another way, the scaffold 100 may function to maintain the nerve ends 26a and 26b facing one another. Said still another way, the scaffold 100 may function to maintain nerve ends 26a and 26b in plane.

Further, in some embodiments, the scaffold 100 may be sufficiently flexible and/or stretchable to permit placement and/or conformance with the sidewall 16 of the form 10, in some embodiments, including in a form 10 having a smaller radius of curvature than the scaffold 100. In various embodiments, the scaffold 100 may be sufficiently flexible to bend or flex with the nerve ends 26a and 26b and with an in situ forming media (e.g., one or both of the growth permissive gel 62a of FIG. 3D and the non-growth permissive gel 62b of FIG. 4A) during motion of a subject after implantation of the scaffold 100 and in-situ forming media. In some embodiments, the scaffold 100 may stretch up to about 50% its length 102 along longitudinal axis L (see FIG. 2). In some embodiments, the scaffold 100 may stretch up to about 35% of its length 102 along longitudinal axis L. In some embodiments, the scaffold 100 may stretch about 5% to about 50%, about 5% to about 35%, about 5% to about 30%, about 5% to about 25%, about 5% to about 20%, about 5% to about 15%, about 10% to about 35%, about 10% to about 20%, about 10% to about 15%, or about 15% to about 20% of its length 102 along longitudinal axis L. In some embodiments, the scaffold 100 may stretch up to about 15% of its length 102 along longitudinal axis L. In some embodiments, the scaffold 100 has a tensile strength of at least about 10 N. In some embodiments, the scaffold 100 has a tensile strength of about 50 N to about 130 N, about 60 N to about 120 N, about 70 N to about 100 N, about 70 N to about 90 N, or about 75 N to about 85 N. In some embodiments, the scaffold 100 has a tensile strength of about 80 N.

In some embodiments, the scaffold 100 may be pre-cut to fit at least a portion of a surface 15 of the sidewall 16 of the form 10 as shown in FIG. 2. In some embodiments, a large scaffold sheet (not shown) may be provided, and a user (e.g., a surgeon or physician) may trim the scaffold sheet to make scaffold 100 having a desired size. For example, an uncut scaffold 100 or scaffold sheet may include pre-scored lines that denote a particular size of scaffold 100 for a particular nerve size. In some embodiments, a scaffold sheet may be provided pre-curled, or the scaffold sheet may be nonplanar. In some embodiments, the scaffold 100 may be thermoformed to fit a curvature of a desired form 10.

In some embodiments, a length 102 of the scaffold 100 may be substantially equal to a length 17 of a sidewall 16 of the form 10. In some embodiments, a length 102 of the scaffold 100 may be undersized relative to a length 17 of the sidewall 16 of the form 10. In some embodiments, a length 102 of the scaffold 100 may be oversized relative to a length 17 of the sidewall 16 of the form 10. In some embodiments, the scaffold 100 may have a thickness (e.g., thickness 104 of FIG. 4B) of about 0.1 mm to about 2 mm, about 0.5 mm to about 1.5 mm, about 0.8 mm to about 1.2 mm, about 0.5 mm to about 1 mm, about 1 mm to about 2 mm, less than or equal to about 1 mm, greater than or equal to about 0.5 mm, or about 1 mm. Because at least part of the in situ forming media (e.g., non-growth permissive gel 62b of FIG. 4A) integrates into the scaffold 100 in some embodiments, the thickness 104 of scaffold 100 allows for a centering of the first and second nerve ends 26a and 26b within the cavity 18 of the form 10 and therefore the production of a substantially even coating of media (e.g., a thickness 624 of the non-growth permissive gel 62b of FIG. 4B) around the circumference of the first and second nerve ends 26a and 26b.

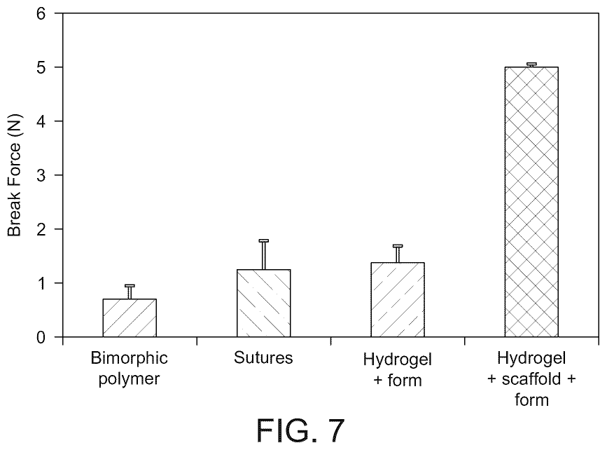

In some embodiments, the scaffold 100 has a particular roughness or provides friction so one or more nerve ends 26a and 26b do not substantially move or pull apart during the introduction of the in situ forming media (e.g., one or both of the growth permissive gel 62a of FIG. 3D or the non-growth permissive gel 62b of FIG. 4A, either of which or both may be a hydrogel) to the form 10. In some embodiments, the scaffold 100 has a static coefficient of friction of about 0.5 to about 1.3, about 0.6 to about 1.2, about 0.7 to about 1.1, or about 0.8 to about 1. In some embodiments, the scaffold 100 functions to distributes stress over a larger area of the nerve ends 26a and 26b. For example, as shown in FIG. 7, when comparing break force (e.g., tensile strength) for a bimorphic polymer (Tissium®), sutures, hydrogel (e.g., in situ forming media) in a form 10, and hydrogel in a form 10 with a scaffold 100, the hydrogel in the form 10 with the scaffold 100 has a significantly higher break force (about 5 N) as compared to the bimorphic polymer (about 0.5 N), sutures (about 1N), or hydrogel in a form (about 1.5 N).

In some embodiments, a gap 50 may be present between the first nerve end 26a and second nerve end 26b when positioned on the scaffold 100. In some embodiments, the gap 50 may be due to an intentional placement of the nerve ends 26a and 26b or a consequence of a type of operation on the nerve ends 26a and 26b. The gap 50 between the first nerve end 26a and the second nerve end 26b may be about 0 mm to about 50 mm in some embodiments. In some embodiments, gap between the first nerve end 26a and the second nerve end 26b may be about 0 mm to about 35 mm, about 0 mm to about 20 mm, about 0 mm to about 15 mm, or about 0 mm to about 10 mm. As shown in FIG. 3B a growth permissive gel 62a as at least part of an embodiment of in situ forming media (i.e., with or without non-growth permissive gel 62b of FIG. 4A) may be introduced to a gap between the first nerve end 26a and second nerve end 26b positioned on the scaffold 100 within the form 10. In some embodiments, due to the physical properties of the growth permissive gel 62a (e.g., high viscosity) and/or the scaffold 100, the growth permissive gel 62a generally does not flow through the scaffold 100 or the scaffold's pores, if any, to reach a sidewall 16 of the form 10. Instead, the growth permissive gel 62a may sit on top of the scaffold 100 and between the nerve ends 26a and 26b in some embodiments.

In some embodiments, the scaffold 100 is wrapped entirely around the circumference of the first and second nerve ends 26a and 26b after delivery of the growth permissive gel 62a between them. In this embodiment, the scaffold 100 is placed as an open sheet at least partially on a surface 15 of a sidewall 16 of the form 10, the first nerve end 26a and second nerve end 26b are placed inside on top of the scaffold 100, the growth permissive gel 62a is introduced between the first and second nerve ends 26a and 26b, and the scaffold 100 is then applied to wrap the scaffold 100 around the first and second nerve ends 26a and 26b circumferentially. Subsequently, a non-growth permissive gel (e.g., non-growth permissive gel 62b of FIG. 4A) can be delivered around the first and second nerve ends 26a and 26b and scaffold 100 to provide additional mechanical strength. In some embodiments, the scaffold 100 is positioned around a part of the circumference of the first and second nerve ends 26a and 26b.

In some embodiments, the scaffold 100 is delivered in two or more pieces or portions around the first and second nerve ends 26a and 26b. For example, a first piece or portion of the scaffold 100 is placed in at least a portion of the cavity 18 of the form 10 (e.g., on a bottom interior surface 15 of the sidewall 16 of the form 10), and the first and second nerve ends 26a and 26b are placed on top of the scaffold 100. After the growth permissive gel 62a is applied to the gap 50 between the two nerve stumps 26a and 26b, a second piece or portion (not shown) of scaffold 100 is then placed on top of one, two, or all of the first and second nerve ends 26a and 26b and growth-permissive gel 62a. In some embodiments, additional in situ forming media (e.g., a non-growth permissive gel 62b of FIG. 4A) can then be introduced into the form 10. In some embodiments, only a first piece or portion of scaffold is positioned in the cavity 18 of the form 10.

Due to the properties of the growth permissive gel 62a and/or the scaffold 100, the growth permissive gel 62a does not integrate into or substantially integrate into the scaffold 100 during a time between the introduction of the growth permissive gel 62a and additional in situ forming media (e.g., a non-growth permissive gel 62b of FIG. 4A). The properties may include, but are not limited to, a high viscosity of the growth permissive gel 62a, and/or the pore size of a pore of the plurality of pores of the scaffold 100. The term “integrate into” is intended to describe a phenomenon of the in situ forming media (e.g., one or both of the growth permissive gel 62a and non-growth permissive gel 62b of FIG. 4A) permeating through the structure of the scaffold 100 (e.g., via pores) and/surrounding the scaffold 100 to form one continuous, combined scaffold 100 and in situ forming media composition. The term “substantially integrate into” is intended to describe a phenomenon of the in situ forming media permeating through and surrounding at least half the thickness (e.g., thickness 104 of FIG. 4B) of the scaffold 100. In some embodiments, the growth permissive gel 62a may sit on top of the scaffold 100 without substantially integrating into the scaffold 100. The time between the introduction of the growth permissive gel 62a and additional in situ forming media (e.g., a non-growth permissive gel 62b of FIG. 4A) may be, for example, less than about 2 hours, less than about 1 hour, less than or about 30 minutes, less than or about 10 minutes, about 15 seconds to about 10 minutes, about 15 seconds to about 5 minutes, about 15 seconds to about 1 minute, about 30 seconds to about 5 minutes, or about 30 seconds to about 1 minute.

FIG. 4A shows the form 10 and scaffold 100 after the introduction of a non-growth permissive gel 62b as at least part of an embodiment of in situ forming media (i.e., with or without growth permissive gel 62a of FIG. 3D) The non-growth permissive gel 62b may be applied around the coaptation site (i.e., around the first and second nerve ends 26a and 26b and any growth permissive gel 62a in between) and may flow around the scaffold 100 to integrate into the scaffold 100 to form a protective barrier at least partially around a circumference of the nerve ends 26a and 26b. For example, the non-growth permissive gel 62b may flow through one or more or a plurality of pores present on the scaffold 100. In some embodiments, the materials of the scaffold 100 and their material properties (e.g., wettability as described herein) may facilitate the integration of the non-growth permissive gel 62b into the scaffold 100 within the without the generation of bubbles. In some embodiments, a visual check for bubble formation may suffice to determine whether the scaffold 100 integrated into non-growth permissive gel 62b.

FIG. 4B shows a cross-section of the embodiment of FIG. 4A along plane B. A thickness 624 of the non-growth permissive gel 62b around the circumference of the nerve ends (e.g., the second nerve end 26b), in some embodiments, may be about 0.05 mm to about 10 mm, about 0.5 mm to about 3 mm, about 0.75 mm to about 2 mm, about 0.75 mm to about 1.25 mm, or about 1 mm. In some embodiments, the thickness 624 of the non-growth permissive gel 62b may vary according to a diameter of the first and second nerve ends 26a and 26b. For example, in embodiments where the first nerve end 26a has a greater diameter than the second nerve end 26b, the thickness 624 of the non-growth permissive gel 62b may be greater around smaller second nerve end 26b.

Because the non-growth permissive gel 62b integrates into the scaffold 100, the the thickness 104 of the scaffold 100 may be included within the thickness 624 of the non-growth permissive gel 62b, and the scaffold 100 may not touch one or more of the form 10 or nerve ends 26a and 26b after the introduction of the non-growth permissive gel 62b. To fit these dimensions of the nerve ends 26a and 26b and in situ forming media (e.g., non-growth permissive gel 62b), the internal diameter 32 of the form 10 may be between about 0.5 mm to about 50 mm, about 1 mm to about 40 mm, about 1.5 mm in to about 30 mm, about 2 mm to about 28 mm, about 3 mm to about 24 mm, about 3 mm to about 12 mm, or about 12 mm to about 24 mm, in some embodiments.

Returning to FIG. 4A, the non-growth permissive gel 62b may be introduced to the form 10 to cover as a substantially continuous mass of material a growth permissive gel 62a between the first and second nerve ends 26a and 26b and up to a coverage length 28a and 28b of the first nerve end 26a and second nerve end 26b, respectively. The coverage length 28a and 28b may be measured from the terminus of the respective nerve end 26a or 26b in the form 10 in a direction along the length of the nerves 26a and 26b away from the gap 50 or a growth permissive gel 62a disposed between the nerve endings 26a and 26b. Generally, for nerve repair procedures, the coverage lengths 28a and 28b may be about 2 mm to about 30 mm, about 3 mm to about 20 mm, about 3 mm to about 15 mm, about 3 mm to about 10 mm, about 4 mm to about 10 mm, about 4 mm to about 9 mm, or about 5 mm to about 8 mm. This amount of coverage by the non-growth permissive gel 62b provides a sufficient protective layer to the first and second nerve ends 26a and 26b and an optional growth permissive gel 62b by remaining adherent to the first and second nerve ends 26a and 26b while providing mechanical strength to hold the first and second nerve ends 26a and 26b in the desired position during the healing process. For nerve repair applications, the length 17 of the form 10 may be between about 6 mm to about 75 mm, about 6 mm and about 60 mm, about 6 mm to about 50 mm, about 6 mm, to about 40 mm, about 6 mm to about 30 mm, about 6 mm to about 25 mm, or about 6 mm to about 20 mm accommodating both the coverage lengths 28a and 28b as well as the distance of any gap between the first and second nerve ends 26a and 26b.

The in situ forming media (e.g., one or both of the growth permissive gel 62a and non-growth permissive gel 62b) may be introduced into the form 10 in a first, relatively flowable state in some embodiments. In some embodiments, this first relatively flowable state allows for a facile introduction of the in situ forming media into the form and in contact with the first nerve end 26a, the second nerve end 26b, and the scaffold 100. After introduction, the in situ forming media may be permitted to undergo a transformation into a second relatively non-flowable state to form a protective barrier surrounding the nerve ends 26a and 26b in some embodiments. For example, the in situ forming media may have a lower viscosity in the first, relatively flowable state than a viscosity of the second, relatively non-flowable state.

Due to the chemical properties of the in situ forming media, the transformation may be pH-initiated, photoinitiated, moisture-initiated, free radical-initiated, oxygen-initiated, or temperature-initiated in some embodiments. In some embodiments, the transformation may occur substantially simultaneously with the introduction of the in situ forming media (e.g., cross-linking occurs within about 0 seconds to about 20 seconds, about 0 seconds to about 15 seconds, about 0 seconds to about 10 seconds, about 5 seconds to about 15 seconds, or about 10 seconds upon introducing the in situ forming media into the form.) For example, a solution of a particular pH may be introduced to the in situ forming media to initiate a pH-initiated transformation. For example, a syringe with a mixing tip may be employed to mix at least one of higher pH solution or a lower pH solution with the in situ forming media during the introduction of the in situ forming media to substantially simultaneously initiate a pH-initiated transformation

For example, a diode emitting a desired wavelength of light (e.g., a UV wavelength) or a fiber optic cable transmitting the desired wavelength may be positioned over or near the in situ forming media to shine light onto the in situ forming media to initiate a photoinitiated transformation. For example, a solution containing water may be introduced to the in situ forming media to initiate a moisture-initiated transformation. For example, a solution containing a free radical polymerization initiator may be introduced into the in situ forming media to initiate a free radical-initiated transformation. For example, a solution containing an initiator may be introduced to the in situ forming media and a tube may transport oxygen gas in effective proximity to or into the in situ forming media to initiate an oxygen-initiated transformation. For example, a heater (e.g., a heat gun) may be pointed at and in effective proximity to the in situ forming media to initiate a temperature-initiated transformation. In some examples, ambient body temperature of a patient is of sufficient temperature to initiate a temperature-initiated transformation. In some embodiments, a syringe with a mixing tip may be employed to mix the in situ forming media with another reagent (e.g., a solution having a particular pH, a solution containing water, etc.) during the introduction of the in situ forming media to the form 10. Once the in situ forming media is in the second relatively non-flowable state, the form 10 may be removed. In some embodiments, the form 10 may be discarded or processed for additional uses.

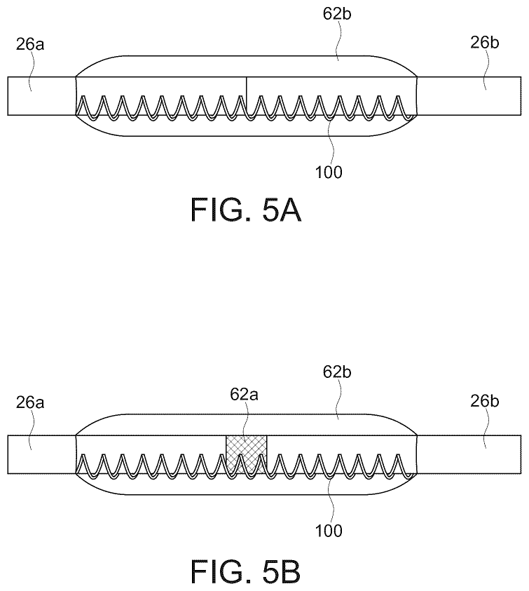

FIGS. 5A and 5B show embodiments of a coaptation site 500 supported by a scaffold 100 and in situ forming media (e.g., non-growth permissive gel 62b with or without growth permissive gel 62a) after the form 10 has been removed. FIG. 5A shows a bottom view of an embodiment in which the first and second nerve ends 26a and 26b are encased or encapsulated by a non-growth permissive gel 62b that has integrated into a scaffold 100. FIG. 5B shows a side view of an embodiment in which the first and second nerve ends 26a and 26b and a growth-permissive gel 62a between the first and second nerve ends 26a and 26b are encased by a non-growth permissive gel 62b that has integrated into a scaffold 100.

In some embodiments, the in situ forming media (e.g., one or both of the growth permissive gel 62a and non-growth permissive gel 62b) is biodegradable. In some embodiments, the in situ forming media remains in situ for between 3 months to about 18 months, about 3 months to about 3 years, about 3 months to about 18 months, about 3 months to about 12 months, about 3 months to about 6 months, about 6 months to about 18 months, or about 6 months to about 12 months before degrading.

The scaffold 100, separately or in combination with the in situ forming media (e.g., one or both of the growth permissive gel 62a and non-growth permissive gel 62b) as described herein in various embodiments, presents a technical solution to at least the following technical problems: (1) fascicular escape; (2) the drooping or sagging of nerve ends 26a and 26b within the form 10; (3) misaligned nerve ends 26a and 26b impeding coaptation; (4) movement of nerve ends 26a and 26b during introduction of in situ forming media; (5) compression or tension on nerve ends 26a and 26b during introduction of in situ forming media (e.g., one or both of the growth permissive gel 62a and non-growth permissive gel 62b); (6) the isolation of a growth permissive gel 62a in a desired location between the nerve ends 26a while permitting a full encapsulation of the coaptation site by the non-growth permissive gel 62b; and (7) fragility of coaptation site without the use of compression or tension.

### In Situ Forming Media

A growth permissive gel, previously described in International Patent Publication No. WO2020010164A1, incorporated herein by reference, provides a region through which nerves and supporting cells, such as Schwann cells can regenerate. In some embodiments, the growth permissive gel 62a may be a gel that does not inhibit nerve regeneration.

In some embodiments, the growth permissive gel 62a may be a hydrogel made of water and one or more of hyaluronic acid (HA), Hyaloglide®, ADCON®-T/N, fibrin (including fibrin crosslinked with thrombin), fibronectin, chitosan, chitosan-coupled alginate, viscous fibronectin, carboxy methyl cellulose (CMC), collagen (including collagen type I), laminin, Corning® PuraMatrix™ peptide hydrogel, heparin sulfate proteoglycans, polyarginine (poly(D, or L, or D,L) arginine), polylysine (poly(D, or L, or D,L) lysine), xyloglucan, polyornithine including poly-L-ornithine, agarose, chitosan-beta-glyverophosphate hydrogels (C/GP) mixtures, poly(N-isopropylacrylamide) (PNIPAAM), poly(Propylene fumarate) (PPF), and various polyethylene glycols (PEGs) as described herein.

In some embodiments, the growth permissive gel 62a may be made of an about 1 wt % to about 10 wt % sodium hyaluronate viscous solution. In some embodiments, the growth permissive gel 62a may be made of an about 1 wt % to about 7.5 wt % sodium hyaluronate viscous solution. In some embodiments, the growth permissive gel 62a may be made of an about 1 wt % to about 5 wt % sodium hyaluronate viscous solution. In some embodiments, the growth permissive gel 62a may be made of an about 2 wt % to about 4 wt % sodium hyaluronate viscous solution. In some embodiments, the growth permissive gel 62a may be made of an about 2.5 wt % to about 3.5 wt % sodium hyaluronate viscous solution. In some embodiments, the growth permissive gel 62a may be made of an about 3 wt % sodium hyaluronate viscous solution. In some embodiments, the growth permissive gel 62a may contain about 100 kDa to about 1500 kDa, about 300 kDa to about 1500 kDa, about 600 kDa to about 1200 kDa, about 800 kDa to about 1000 kDa, or about 900 kDa HA polymer chains.

In some embodiments, the growth permissive gel 62a features an interpenetrating network of HA and photocrosslinkable glycidyl methacrylate hyaluronic acid (GMHA). In some embodiments, the growth permissive gel 62a includes of fibrin at a concentration of about 9 mg/mL to about 50 mg/mL. In some embodiments, the growth permissive gel 62a includes collagen at a concentration of about 1.28 mg/mL. In some embodiments, the growth permissive gel 62a is about 0.001 wt % to about 20 wt % collagen. In some embodiments, the growth permissive gel 62a is about 3 wt % to about 6 wt % collagen. In some embodiments, the growth permissive gel 62a is made of agarose at about 0.5% to about 1% w/v. In some embodiments, poly-L-arginine and/or poly-L-lysine may be individually about 0.001 wt % to about 10 wt % or about 0.01 wt % to about 0.1 wt % of the growth permissive gel 62a. In some embodiments, laminin may be about 0 wt % to about 5 wt % or about 0 wt % to about 0.5 wt % of the growth permissive gel 62a.

PEG hydrogels for the growth permissive gel 62a, in some embodiments, may feature PEG-NHS esters cross-linked with hydrolytically labile ester bonds, including but not limited to PEG-succinimidyl succinate (PEG-SS), PEG-succinimidyl glutarate ester (PEG-SG), PEG-succinimidyl azelate (PEG-SAZ), PEG-succinimidyl adipate (PEG-SAP), PEG-amines, or trilysines. The PEG species may be multi-armed in some embodiments.

In some embodiments, a PEG hydrogel for use as the growth permissive gel 62a may feature a growth promoting additive including, but not limited to, HA, collagen (including collagen type I), laminin, fibrin, adhesive ligands, polyarginine (poly(D, or L, or D,L) arginine), polylysine (poly(D, or L, or D,L) lysine), and various peptides. In some embodiments, an additive of collagen is about 0.01 wt % to about 5 wt %, about 0.3 wt % to about 0.5 wt %, or about 3 wt % to about 6 wt % of the growth permissive gel 62a. In some embodiments, an additive of collagen is present in the growth permissive gel 62a at a concentration of about 1.28 mg/mL. In some embodiments, an additive of laminin makes is about 0 wt % to about 0.5 wt % of the growth permissive gel 62a, and the laminin is at a concentration of about 4 mg/mL. In some embodiments, an additive of fibrin has a strength of about 2.1 kPa and is present in the growth permissive gel 62a a concentration of about 9 mg/mL to about 50 mg/mL. In some embodiments, the poly-L-arginine and/or poly-L-lysine are individually about 0.001 wt % to about 10 wt % or about 0.01 wt % to about 0.1 wt % of the growth permissive gel 62a.

In some embodiments, the growth promoting additive may be a matrix component, such as a fiber, rod, filament, sponge, bead, microsphere, microsphere, nanoparticle, or liposome to facilitate nerve regeneration through the growth permissive gel 62a. The matrix component to gel volume ratio is, in some embodiments, about 1:10 or about 1:50 such that the nerve ends 26a and 27b have sufficient space to grow through the growth permissive gel 62a along the fibers of the matrix. In some embodiments, the fibers of the matrix are about 50 μm to about 1000 μm in diameter or about 50 μm to about 100 μm in diameter. In some embodiments, the fibers, filaments, or rods of the matrix may be made of chitosan, polycaprolactone (PCL), PCL missed with gelatin, polylactic acid (including PLA, PLLA, and PDLA), polyglycolic acid, collagen-glycosaminoglycan (GAG), fibrin, HA, polyamide, polyacrylonitrile-co-methacrylate, polyacrylonitrile-methyl acrylate, PGA, PLGA, poly(3-hydroxybutyrate-co-3-hydroxyvalerate) (PHBV)/PLGA blends, polypropylene (PP), and PEG. In some embodiments, the matrix component may be coated with laminin or functionalized with a positive charge.

The growth permissive gel 62a of the in-situ forming media is largely made of water with a solid material content between about 0 wt % to about 20 wt %, about 1 wt % to about 10 wt %, or about 1 wt % to about 5 wt %, in some embodiments. In some embodiments, the growth permissive gel 62a may have high viscosity during a first, relatively flowable state. For example, the growth permissive gel 62a may have a viscosity of about 50 kcPs to about 1000 kcPs, about 100 kcPs to about 750 kcPs, about 250 to about 500 kcPs, or about 500 kcPS. In some embodiments, the growth permissive gel 62a is hydrophilic. In some embodiments, the growth permissive gel 62a is hydrophobic. In some embodiments, the growth permissive gel 62a is amphipathic. In some embodiments, the growth permissive gel 62a is positively charged or neutral. In some embodiments, the growth permissive gel 62a is less stiff than a non-growth permissive gel (e.g., non-growth permissive gel 62b of FIG. 4A). A gel stiffness of the growth permissive gel 62a may be less than about 800 dynes/cm² or less than about 200 dynes/cm² in some embodiments. In some embodiments, the growth permissive gel 62a may have an elasticity of less than about 0.2 MPa, less than about 0.1 MPa, or less than about 1.5 kPa. In some embodiments, the growth permissive gel 62a swells in volume after introduction by less then 20% or between 5% and 20%. Optionally, a color additive may be included in the growth permissive gel 62a in some embodiments, such as FD&C No. 1, Brilliant Blue FCF, or Brilliant Blue BBG.

For example, a growth permissive gel 62a may feature HA and water with additives of polylysine, collagen, and laminin, in which the polylysine is about 0.01 wt % to about 0.1 wt %, the collagen is about 3 wt % to about 6 wt %, and the laminin is about 0 wt % to about 0.5 wt % of the growth permissive gel 62a.

To allow for a greater case of introduction into a form 10 (i.e., into a gap 50 between nerve ends 26a and 26b of FIG. 3A), in some embodiments, the growth permissive gel 62a, as part of in situ forming media, may be introduced as a precursor solution having different mechanical properties than it has when later transformed (e.g., polymerized) into a hydrogel. For example, as a precursor solution, the growth permissive 62a may have a lower viscosity than when polymerized into a hydrogel, in some embodiments. After delivery into the form 10 as a precursor solution (i.e., in a first, relatively flowable state), the precursor solution may be transformed into a polymerized hydrogel (i.e., in a second, relatively non-flowable stat). In various embodiments, the transformation may be pH initiated, photo initiated, moisture initiated, oxygen initiated, or temperature initiated. A transformation of the growth permissive gel 62a may occur before, after, or simultaneously with a transformation of a non-growth permissive gel 62b as part of in situ forming media in various embodiments.

A non-growth permissive gel 62b, also previously described in International Patent Publication No. WO2020010164A1 which is incorporated herein by reference, forms a conformable barrier to inflammatory cell infiltration and aberrant nerve outgrowth during the nerve repair process. For example, after encasing the distal nerve stump (e.g., the second nerve end 26b), the distal nerve stump may release growth factors that provide a trophic concentration gradient for proximal regenerating nerves (e.g., the first nerve end 26a) to follow during regrowth. By preventing the diffusion of these growth factors away from the coaptation site with a non-growth permissive gel and concentrating them in the growth permissive gel, the speed and extent of regenerating nerves reaching the distal stump may increase in some embodiments.

In some embodiments, the non-growth permissive gel 62b may include water and at least one of agarose, alginate, polyvinyl alcohol, poly(acrylamide), hyaluronic acid, and polyethylene glycol (PEG). In some embodiments, the non-growth permissive gel 62b may include polyethylene glycol (PEG) and water. In some embodiments, the non-growth permissive gel 62b is a PEG hydrogel. In some embodiments, the PEG hydrogel is cross-linked. In some embodiments the PEG hydrogel is multi-armed. In some embodiments, the PEG hydrogel may be crosslinked by urea, urethane, and/or carbamate groups. For example, the PEG hydrogel may be a PEG ester, PEG isocyanate, or a PEG succinimidyl carbonate (PEG-SC). In some embodiments, the PEG hydrogel may be a 4-arm or 8-arm PEG-SC with molecular weight of about 5 to about 40 kDa and arm lengths of about 1 kDa to about 3 kDa. In some embodiments, the PEG hydrogel may be a 4-arm or 8-arm PEG-amine with molecular weight of about 5 kDA to about 40 kDa. In 4-arm PEG-SC of about 10 kDa. In some embodiments, the PEG hydrogel may be an 8-arm PEG-SC of about 20 kDa. In some embodiments, the PEG hydrogel may be crosslinked with trilysine amine. In some embodiments, the PEG hydrogel may be a PEG-maleimide-PEG-thiol, a PEG-acrylate, a PEG-norbornene-PEG-thiol, or a PEG-polycaprolactone-n-hydroxysuccinimide ester.

Generally, the structure of multi-armed PEGs described herein is

C-[(PEG)ₙ-M-L-F]ₘ
where
C is the core structure of the multi-armed PEG;
n is the number of repeating units of PEG in each arm. In some embodiments, n may be about 25 to about 60 units;
M is an optional modifier group as described herein;
L is a cleavable or non-cleavable linker (e.g., ester, urethane, amide, urea, carbamate, carbonate, thiourea, thioester, disulfide, hydrazone, oxime, imine, amidine, triazole, and thiol/maleimide, etc.);
F is a reactive functional group for covalent cross-linking (e.g., maleimide, thiol or protected thiol, alcohols, acrylates, acrylamides, amines, protected amines, carboxylic acids or protected carboxylic acids, azides, alkynes, 1.3-dienes, furans, alpha-halocarbonyls, and N-hydroxysuccinimidyl, N-hydroxysulfosuccinimidyl, or nitrophenyl esters or carbonates; and
m is the number of PEG arms (e.g., 2, 3, 4, 6, 8, 10, etc.)

A variety of core structures C are available in various embodiments. Examples of core structures C include, but are not limited to, glycerol, pentaerythritol, tripentaerithytol, d-sorbitol, and hexaglycerol.

The optional modifier group M may be, in some embodiments, an electron donating group (e.g., an aliphatic chain integrated into the PEG backbone) to increase the cleavability of linker L. In some embodiments, M is an electron withdrawing group (e.g., an aromatic group integrated into the PEG backbone) to decrease the cleavability of linker L.

In some embodiments, different PEG polymers may be crosslinked together. For example, a PEG-SC may be crosslinked with a PEG-amine. In some embodiment, a PEG succinimidyl glutaramide (PEG-SGA) may be cross-linked with a PEG-amine.

In some embodiments, the PEG arm of a multi-armed PEG hydrogel may contain a block of another polymer to modulate the degradation time of the hydrogel in situ. For example, the PEG arms may include low molecular weight regions of, e.g., polyester, polycaprolactone, polylactic acid, polyglycolic acid, polyurethane, polyhydroxyalkanoates, poly(ethylene adipate), and aliphatic diisocyanates such as isophorone diisocyanate or L-lysine ethyl ester diisocyanate. In some embodiments, the non-growth permissive gel 62b may include PEG-polycaprolactone-n-hydroxysuccinimide ester.

In some embodiments, a PEG hydrogel of the non-growth permissive gel 62b may include additional excipients to modulate the material properties of the non-growth permissive gel 62b, including but not limited to mechanical strength, density, surface tension, flowability, and in vivo persistence. Examples include, but are not limited to, amphiphilic excipients such as vitamin E TPGS, poloxamer (e.g., Pluronic®), low molecular weight polyesters such as caprolactone, and solvents such as ethanol. In some embodiments, ethanol improves the elasticity of the non-growth permissive gel 62b.

In some embodiments, the non-growth permissive gel 62b has an elastic modulus of about 70 kPa to about 120 kPa, about 80 kPa to about 110 kPa, about 90 kPa to about 110 kPa, about 90 kPa to about 100 kPa, or about 100 kPa. In some embodiments, the non-growth permissive gel 62b has a linear compressive modulus of at least about 10 kPa, at least about 30 kPa, or at least about 50 kPa. In some embodiments, the non-growth permissive gel 62b swells less than about 30% of its original volume, about 5% to about 30% of its original volume, or about 5% to about 25% of its original volume. In some embodiments, the non-growth permissive gel 62b has a low to medium viscosity in its first, relatively flowable state. In some embodiments, the non-growth permissive gel 62b, in its first, relatively flowable state, has a viscosity less than about 10 kcP, less than 900 cP, about 2 cP to about 900 cP, about 300 cP to about 900 cP, about 2 cP to about 100 cP, about 2 cP to about 20 cP, about 2 cP to about 5 cP.

In some embodiments, the non-growth permissive gel 62b may include a viscosity modifier like a natural hydrocolloid, semisynthetic hydrocolloid, synthetic hydrocolloid, or a clay. Examples of natural hydrocolloids include, but are not limited to, acacia, tragacanth, alginic acid, alginate, karaya, guar gum, locust bean gum, carrageenan, gelatin, collagen, hyaluronic acid, dextran, starch, xanthan gum, galactomannans, konjac mannan, gum tragacanth, chitosan, gellan gum, methoxyl pectin, agar, gum Arabic, and dammar gum. Examples of semisynthetic hydrocolloids include, but are not limited to, methylcellulose, carboxymethylcellulose, ethyl cellulose, hydroxyethyl cellulose, and hydroxypropylmethyl cellulose. Examples of synthetic hydrocolloids include, but are not limited to, polyethylene glycol, polyacrylic acid, polyvinyl alcohol, polyvinyl pyrrolidone, polyglycerol, and polyglycerol polyricinoleate. Examples of clays include, but are not limited to, magnesium aluminum silicate (e.g., Vecgum®), bentonite, and attapulgite.

In some embodiments, the non-growth permissive gel 62b is about 1 wt % to about 25 wt % solid content. In some embodiments, the non-growth permissive gel 62b is about 1 wt % to about 20 wt % solid content. In some embodiments, the non-growth permissive gel 62b is about 1 wt % to about 15 wt % solid content. In some embodiments, the non-growth permissive gel 62b is about 3 wt % to about 20 wt % solid content. In some embodiments, the non-growth permissive gel 62b is about 3 wt % to about 15 wt % solid content. In some embodiments, the non-growth permissive gel 62b is about 5 wt % to about 15 wt % solid content. In some embodiments, the non-growth permissive gel 62b is about 8 wt % to about 12 wt % solid content.

To allow for a greater case of introduction into a form 10 (i.e., to produce coverage lengths 28a and 28b around the circumference of nerve ends 26a and 26b), in some embodiments, the non-growth permissive gel 62b, as part of in situ forming media, may be introduced as a precursor solution having different mechanical properties than it has when later transformed (e.g., polymerized) into a hydrogel. For example, as a precursor solution, the non-growth permissive 62b may have a lower viscosity than when polymerized into a hydrogel, in some embodiments. After delivery into the form 10 as a precursor solution (i.e., in a first, relatively flowable state), the precursor solution may be transformed into a polymerized hydrogel (i.e., in a second, relatively non-flowable stat). In various embodiments, the transformation may be pH initiated, photo initiated, moisture initiated, oxygen initiated, or temperature initiated. A transformation of the non-growth permissive gel 62b may occur before, after, or simultaneously with a transformation of a growth permissive gel 62a as part of in situ forming media in various embodiments.

### Methods

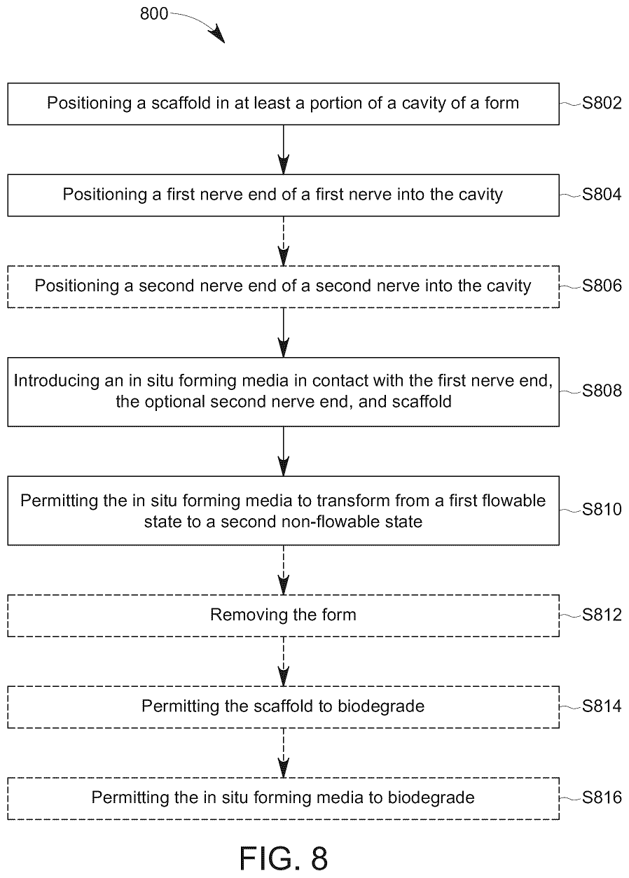

As shown in FIG. 8, the disclosure herein includes for a method 800 for facilitating nerve growth. In some embodiments, the method 800 includes, positioning a scaffold in at least a portion of a cavity of a form in block S802; positioning a first nerve end of a first nerve in block S804; optionally positioning a second nerve end of a second nerve, into the cavity in block S806; introducing an in situ forming media in contact with the first nerve end, the optional second nerve end, and scaffold in block S808; permitting the in situ forming media to transform from a first flowable state to a second non-flowable state in block S810; optionally removing the form in block S812; optionally permitting the scaffold to biodegrade in block S814; and optionally permitting the in situ forming media to biodegrade in block S816. The method 800 is used for surgically treating nerves, in some embodiments, but can be additionally or alternatively used for any suitable application, clinical or otherwise.

In block S802, the method 800 includes positioning a scaffold in at least a portion of a cavity of a form. In some embodiments, the scaffold may be the scaffold 100 as described herein into the cavity 18 of form 10 as described herein. In some embodiments, the scaffold 100 may be at least partially positioned on a surface of a sidewall of the form. In some embodiments, the scaffold may be positioned in the form as depicted in FIG. 2.

In block S804, the method 800 includes positioning a first nerve end of a first nerve into the cavity. In some embodiments, the first nerve end of the first nerve is the first nerve end 26a of FIG. 3A. In some embodiments, the first nerve end is positioned at least partially in contact with the scaffold within the cavity. In some embodiments, the method 800 further includes offsetting, using the scaffold, the first nerve end from a sidewall (e.g., sidewall 16 of FIG. 1A) of the form. In some embodiments, the first nerve end is positioned through a nerve guide (e.g., nerve guide 22a of FIG. 1A.)

In block S806, the method 800 optionally includes positioning a portion of a second nerve end into the cavity. In some embodiments, the second nerve end is the second nerve end 26b of FIG. 3B. In some embodiments, a connection target (e.g., connection target 26c of FIG. 3C) is at least partially placed into the cavity instead of a second nerve end. In some embodiments, the second nerve end or connection target is positioned in contact with the scaffold within the cavity. In some embodiments, the method 800 further includes offsetting, using the scaffold, the second nerve end or connection target from a sidewall (e.g., sidewall 16 of FIG. 1A) of the form. In some embodiments, the second nerve end or connection target is positioned through nerve guides 22a and 22b of FIG. 1A. In some embodiments, the first nerve end and the second nerve end or connection target are positioned as depicted in FIGS. 3B and 3C, respectively.

In block S808, the method 800 includes introducing an in situ forming media in contact with the first nerve end, the optional second nerve end or connection target, and scaffold. In some embodiments, the introducing the in situ forming media includes introducing a growth permissive gel and a non-growth permissive gel. In some embodiments, the growth permissive gel is the growth permissive gel 62a of FIG. 3D and as described herein. In some embodiments, the growth permissive gel is introduced into a gap between the first nerve end and the second nerve end or connection target. In some embodiments, the gap is about 0 mm to about 20 mm in length between the first nerve end and the second nerve end. In some embodiments, the gap is about 0 mm to about 10 mm in length between the first nerve end and the second nerve end. In some embodiments, the growth permissive gel does not integrate into the scaffold. In some embodiments, the non-growth permissive gel is the non-growth permissive gel 62b of FIG. 4A as described herein. In some embodiments, the scaffold produces friction on the first nerve end and second nerve end effective to hold the first nerve end and the second nerve end in a static position during the introducing of the in situ forming media. In some embodiments, the introducing the in situ forming media includes introducing a growth permissive gel between a first nerve end and a second nerve end. In some embodiments, the introducing the in situ forming media includes introducing a non-growth permissive gel around a nerve end, or around a first nerve end and a second nerve end for coaptation.

In block S810, the method 800 includes permitting the in situ forming media to transform, from a first flowable state to a second, non-flowable state. As described herein, the transformation of the in situ forming media (e.g., one or both of a growth permissive gel 62a of FIG. 3D and a non-growth permissive gel 62b of FIG. 4A) may be pH initiated, photo initiated, moisture initiated, free radical-initiated, oxygen initiated, or temperature initiated in various embodiments. A transformation of the non-growth permissive gel 62b may occur before, after, or simultaneously with a transformation of a growth permissive gel 62a as part of in situ forming media in some embodiments.

In block S812, the method 800 optionally includes removing the form. In some embodiments, the form is made of a nonadherent, nondegradable material, such as, but not limited to, medical-grade silicone, sufficiently flexible to be peeled or popped off from the in situ forming material.

In block S814, the method 800 optionally includes permitting the scaffold to biodegrade. In some embodiments, the scaffold degrades in about 3 months to about 6 months, about 1 month to about 6 months, about 1 month to about 3 months, about 1 month to about 2 months. In some embodiments, the scaffold degrades in situ in about 6 weeks to about 8 weeks.

In block S816, the method 800 optionally includes permitting the in situ forming media to biodegrade. In some embodiments, the in situ forming media degrades in about 3 months to about 18 months, about 3 months to about 3 years, about 3 months to about 18 months, about 3 months to about 12 months, about 3 months to about 6 months, about 6 months to about 18 months, or about 6 months to about 12 months.

### Experimental Procedures

Various experimental procedures were performed to measure material and mechanical properties reported herein of the scaffold (e.g., scaffold 100 of FIG. 2), the in situ forming media (e.g., one or both of the growth permissive gel 62a of FIG. 3D and the non-growth permissive gel 62b of FIG. 4A), separately, in combination, or additionally in combination with model rat sciatic nerves.

### Scaffold Wettability

The wettability of a scaffold (e.g., scaffold 100 of FIG. 2) was tested via a droplet penetration test on an example scaffold. The example scaffold was made of warp knitted PGA fiber. A droplet penetration test measures the time required for a droplet of deionized water placed in contact with a dry scaffold to sufficiently enter the scaffold material such that the droplet has no remaining volume physically raised above the scaffold's surface. A 22-gauge needle was positioned with its tip 2 cm above the surface a dry scaffold. A single droplet was dispensed and observed with times recorded in seconds(s). Results are reported in Table A below.

TABLE A

| Sample No. | Droplet Penetration Time (s) |
|---|---|
| Sample 1 | 0.52 |
| Sample 2 | 0.32 |
| Sample 3 | 0.60 |
| Sample 4 | 1.1 |
| Sample 5 | 0.5 |
| Sample 6 | 0.34 |
| Average | 0.56 |

A wettability of this magnitude (i.e., is “substantially wettable” as defined herein) is a technical solution that assists the integration of in situ forming media (e.g., a non-growth permissive gel 62b of FIG. 4A) into the scaffold (e.g., scaffold 100 of FIG. 2). The non-growth permissive gel 62b may flow around the scaffold 100 and polymerize into a second, relatively non-flowable state without the generation of bubbles in some embodiments.

As shown in Table A, wettability of the scaffold, as described by a droplet penetration test, may be between about 0.2 seconds to about 3 seconds, about 0.3 seconds to about 3 seconds, about 0.3 seconds to about 2 seconds, about 0.3 seconds to about 1.5 seconds, about 0.3 seconds to about 1 second, about 0.3 seconds to about 0.75 seconds, or less than about 1 second.

### Scaffold Friction

The coefficient of static friction of an example scaffold (e.g., scaffold 100 of FIG. 2) was measured via a TA.XTplusC Texture Analyzer operating on a segment of model chicken sciatic nerves placed onto the sample scaffold. The ratio of force required to displace the nerve segments was compared to the mass of the nerve segments results the coefficient of static friction for the material pairing. The example scaffold was made of warp knitted PGA fiber. Experimental results yielded a coefficient of static friction of about 0.8 to about 1 for the model chicken sciatic nerves. In some embodiments, because the scaffold has a coefficient of static friction of about 0.8 to about 1, the scaffold may hold or retain the nerve or nerves (e.g., nerve ends 26a and 26b of FIG. 3A) in a desired position during the delivery of the in situ forming media (e.g., non-growth permissive gel 62b of FIG. 4A with or without growth permissive gel 62a of FIG. 3D). In other words, because of the technical solution of the friction provided by the scaffold, the nerve or nerve ends do not substantially move or pull apart during the introduction of the in situ forming media.

As shown by the results described above, the scaffold may have a static coefficient of friction of about 0.5 to about 1.3, about 0.6 to about 1.2, about 0.7 to about 1.1, or about 0.8 to about 1 in interaction with a nerve.

### Scaffold Degradation

Scaffold (e.g., scaffold 100 of FIG. 2) degradation times were measured by observation in vitro (i.e., in a PBS buffer) and in situ with in situ forming media (e.g., a growth permissive gel 62a of FIG. 3D and a non-growth permissive gel 62b of FIG. 4A) around a model rat sciatic nerve. The example scaffold was made of warp knitted PGA fiber. The example growth permissive gel was a 900 kDa 3 wt % HA hydrogel and the example non-growth permissive gel was a 10 wt % PEG hydrogel of PEG-amine and PEG-ester copolymer. The scaffolds were observed until only small particles remained visible. In vitro, the example scaffold degraded in about 8 weeks. In situ, the example scaffold degraded in about 6 weeks. As shown by the results described above, the scaffold may degrade in some embodiments, in about 1 month to about 6 months, about 3 months to about 6 months, about 1 month to about 3 months, about 1 month to about 2 months, or about 6 weeks to about 8 weeks. The technical solution of a degradation rate of about 6 weeks to about 8 weeks, in some embodiments, allows the scaffold to provide mechanical strength to the coaptation site during the nerve healing process and subsequently disappear without further surgery to remove the scaffold.

### Non-Growth Permissive Gel Elastic Modulus

Cylindrical plugs of an example non-growth permissive gel (e.g., the non-growth permissive gel 62b of FIG. 4A) in a second, relatively non-flowable state, were tested to calculate an elastic modulus for the non-growth permissive gel. The plugs were 7 mm in diameter and 8 mm by height and compressed by a texture analyzer to a 17% strain. The modulus was calculated from the linear range of the stress strain curve. Results are reported in Table B below.

TABLE B

| Sample No. | Elastic Modulus (kPa) |
|---|---|
| Sample 1 | 99.257 |
| Sample 2 | 95.413 |
| Sample 3 | 106.091 |
| Sample 4 | 74.56 |
| Sample 5 | 100.26 |
| Sample 6 | 102.345 |
| Sample 7 | 93.992 |
| Sample 8 | 101.92 |
| Sample 9 | 95.416 |
| Average | 96.58 |

Non-growth permissive gel having an elastic modulus of about 70 kPa to about 120 kPa, in some embodiments, is a technical solution that allows the in situ forming media to flex and bend with the subsequent motion of the nerve or nerve ends (e.g., nerve ends 26a and 26b of FIG. 3A) after the introduction of the in situ forming media without deleteriously compressing or constricting the nerve or nerve ends.

As shown in Table B, the non-growth permissive gel may have an elastic modulus of about 70 kPa to about 120 kPa, about 80 kPa to about 110 kPa, about 90 kPa to about 110 kPa, about 90 kPa to about 100 kPa, or about 100 kPa.

### Nerve Pull Test

Model chicken sciatic nerves were subjected to the devices and methods as disclosed herein and tested for tensile strength of the coaptation site. Sciatic nerves of about 3 mm in diameter were severed and reconnected using an example form (e.g., form 10 of FIG. 1A), an example scaffold (e.g., scaffold 100 of FIG. 2), and an example in situ forming media (e.g., a growth permissive gel 62a of FIG. 3D in combination with a non-growth permissive gel 62b of FIG. 4A) as described herein. The example scaffold was made of warp knitted PGA fiber. The example growth permissive gel was a 900 kDa 3 wt % HA hydrogel and the example non-growth permissive gel was a PEG hydrogel of PEG-amine and PEG-ester copolymer. The exposed ends of the nerves (i.e., ends not covered by in situ forming media) were secured to sandpaper with glue to provide a grip for a texture analyzer. The texture analyzer then pulled the nerve ends apart at a rate of 40 mm/min, and the peak force before coaptation failure was recorded. Coaptation failure in each example featured a detachment of the nerve from the in situ forming media. This differs from the experimental results reported in FIG. 7 which replaced the model nerves with a surrogate material with greater adhesion design to measure the force required to tear the in situ forming media and other materials as described herein. Results for the nerve pull test are reported in Table C below.

TABLE C

| Sample No. | Failure Tension (N) |
|---|---|
| Sample 1 | 0.72 |
| Sample 2 | 1.059 |
| Sample 3 | 0.50 |
| Sample 4 | 1.027 |
| Sample 5 | 0.925 |
| Sample 6 | 0.91 |
| Sample 7 | 0.84 |
| Sample 8 | 0.82 |
| Sample 9 | 1.372 |
| Sample 10 | 2.26 |
| Average | 1.04 |

A coaptation failure tension of about 0.25 N to about 2.5 N, in some embodiments, is a technical solution that provides a secure fixation of the nerve or nerve ends (e.g., nerve ends 26a and 26b of FIG. 3A) on the scaffold within the in situ forming media without deleteriously compressing, constricting, or tensioning the nerve or nerve ends.

As shown in Table C, the devices herein may have a coaptation failure tension of about 0.25 N to about 2.5 N, about 0.5 N to about 2 N, about 0.5 N to about 1.5 N, about 0.5 N to about 1 N, about 0.75 N to about 1.25 N, to about 1 N.

### EXAMPLES

Some aspects described herein relate to the following numbered alternatives:
1. A method of facilitating nerve growth, the method comprising: positioning a scaffold in at least a portion of a cavity defined by a form; positioning a first nerve end of a first nerve at least partially onto the scaffold in the cavity; introducing an in situ forming media in contact with the first nerve end and the scaffold; and permitting the in situ forming media to undergo a transformation from a first, relatively flowable state to a second, relatively non-flowable state to form a protective barrier surrounding the first nerve end.
2. The method of alternative 1, wherein the method further comprises, positioning a second nerve end of a second nerve at least partially onto the scaffold in the cavity, wherein the in situ forming media is further in contact with the second nerve end, and wherein the in situ forming media further forms a protective barrier surrounding the first nerve end and the second nerve end.
3. The method of alternative 1, wherein the method further comprises, positioning a connection target at least partially onto the scaffold in the cavity, wherein the in situ forming media is further in contact with the connection target, and wherein the in situ forming media further forms a protective barrier surrounding the first nerve end and the connection target.
4. The method of alternative 3, wherein the connection target is a prosthesis attachment point or a portion of non-nervous tissue.
5. The method of alternative 2, wherein the introducing an in situ forming media comprises introducing a growth permissive gel and a non-growth permissive gel.
6. The method of alternative 5, wherein the growth permissive gel is introduced into a gap between the first nerve end and the second nerve end.
7. The method of alternative 6, wherein the gap is about 0 mm to about 20 mm in length between the first nerve end and the second nerve end.
8. The method of alternative 6, wherein the gap is about 0 mm to about 10 mm in length between the first nerve end and the second nerve end.
9. The method of alternative 5, wherein the growth permissive gel does not integrate into the scaffold.
10. The method of alternative 5, wherein the non-growth permissive gel integrates into the scaffold.
11. The method of alternative 5, wherein the growth permissive gel comprises hyaluronic acid and water.
12. The method of alternative 5, wherein the growth permissive gel further comprises chitosan, polylysine, collagen, fibronectin, poly-L-ornithine, and laminin.
13. The method of alternative 12, wherein the growth permissive gel is about 0.01 wt % to about 0.1 wt % polylysine, about 3 wt % to about 6 wt % collagen, and about 0 wt % to about 0.5 wt % laminin.
14. The method of alternative 5, wherein the non-growth permissive gel comprises polyethylene glycol and water.
15. The method of alternative 14, wherein the non-growth permissive gel is about 3 wt % to about 15 wt % solid content.
16. The method of alternative 2, wherein the introducing the in situ forming media provides a coverage length of about 3 mm to about 20 mm on the first nerve end and the second nerve end.
17. The method of alternative 2, wherein the introducing the in situ forming media provides a coverage length of about 3 mm to about 10 mm on the first nerve end and the second nerve end.
18. The method of alternative 2, wherein the introducing the in situ forming media provides a coverage length of about 5 mm to about 8 mm on the first nerve end the second nerve end.
19. The method of alternative 1, wherein a thickness of the scaffold is less than or equal to about 1 mm.
20. The method of alternative 1, wherein a thickness of the scaffold is about 0.8 mm to about 1.2 mm.
21. The method of alternative 1, wherein a thickness of the scaffold is about 1 mm.
22. The method of alternative 1, wherein the scaffold has a plurality of pores.
23. The method of alternative 22, wherein the plurality of pores of the scaffold are each about 0.8 mm2 to about 1.2 mm2 in cross-sectional area.
24. The method of alternative 1, wherein the scaffold is biodegradable.
25. The method of alternative 24, wherein the scaffold biodegrades in about 1 month to about 6 months.
26. The method of alternative 25, wherein the scaffold biodegrades in about 6 weeks to about 8 weeks.
27. The method of alternative 1, wherein the scaffold comprises a technical textile.
28. The method of alternative 1, wherein the scaffold comprises a knitted material.
29. The method of alternative 28, wherein the scaffold comprises a warp knitted material.
30. The method of alternative 1, wherein the scaffold comprises a woven material.
31. The method of alternative 1, wherein the scaffold comprises a natural monofilament, polydioxanone, poly(lactic-co-glycolic acid), poliglecaprone, polyglactin, polyglycolic acid, polyglycolide fiber, polylactide fiber, collagen, alginate, chitosan, cellulose, or carboxymethylcellulose.
32. The method of alternative 31, wherein the scaffold comprises polyglycolide fiber.
33. The method of alternative 32, wherein the scaffold comprises warp knitted polyglycolide fiber.
34. The method of alternative 1, wherein the scaffold is hydrophilic.
35. The method of alternative 1, wherein the first nerve end is positioned in contact with the scaffold within the cavity.
36. The method of alternative 1, further comprising offsetting, using the scaffold, the first nerve end from a sidewall of the form.
37. The method of alternative 1, wherein the scaffold is configured to produce friction on the first nerve end effective to hold the first nerve end in a substantially static position during the introducing of the in situ forming media.
38. The method of alternative 2, wherein the first nerve end and the second nerve end are positioned in contact with the scaffold within the cavity.
39. The method of alternative 2, further comprising offsetting, using the scaffold, the first nerve end and the second nerve end from a sidewall of the form.
40. The method of alternative 2, wherein the scaffold is configured to produce friction on the first nerve end and the second nerve end effective to hold the first nerve end and the second nerve end in a static position during the introducing of the in situ forming media.
41. The method of alternative 1, wherein the scaffold has a static coefficient of friction of about 0.8 to 1.
42. The method of alternative 1, wherein the scaffold has an elastic modulus of at least about 1 kPa.
43. The method of alternative 42, wherein the scaffold has an elastic modulus of about 15 kPa to about 45 kPa.
44. The method of alternative 1, wherein the scaffold has a tensile strength of at least about 10 N.
45. The method of alternative 44, wherein the scaffold has a tensile strength of about 70 N to about 100 N.
46. The method of alternative 1, wherein the in situ forming media is biodegradable.
47. The method of alternative 46, wherein the in situ forming media biodegrades in about 3 months to about 3 years.
48. The method of alternative 47, wherein the in situ forming media biodegrades in about 3 months to about 6 months.
49. The method of alternative 1, further comprising removing the form.
50. A system for creating a protective barrier in situ around a nerve end or nerve to nerve junction, the system comprising: a form comprising: a sidewall defining a cavity and a top opening for accessing the cavity, the top opening configured to receive a first nerve end; and a scaffold at least partially positioned on a bottom interior surface of the sidewall of the form such that the first nerve end is offset from the bottom interior surface of the sidewall when the first nerve end is positioned on the scaffold within the cavity.
51. The system of alternative 50, wherein the top opening is configured to receive a second nerve end, and wherein the second nerve end is offset from the bottom interior surface of the sidewall when the second nerve end is positioned on the scaffold within the cavity.
52. The system of alternative 50, wherein the top opening is configured to receive a connection target, and wherein the connection target is offset from the bottom interior surface of the sidewall when the connection target is positioned on the scaffold within the cavity.
53. The system of alternative 52, wherein the connection target is a prosthesis attachment point or a portion of non-nervous tissue.
54. The system of alternative 50, wherein the top opening has a first cross-sectional area being less than a second cross-sectional area of the cavity.
55. The system of alternative 50, wherein the form further comprises a first nerve guide comprising a first surface at least partially defining a first side access through the sidewall for positioning a first nerve end in the cavity, the first surface being on a first end of the form at a first elevated position relative to a bottom interior surface of the sidewall.
56. The system of alternative 51, further comprising a second nerve guide comprising a second surface at least partially defining a second side access through the sidewall for positioning a second nerve end in the cavity, the second surface being on a second end of the form at a second elevated position relative to a bottom interior surface of the sidewall.
57. The system of alternative 50, further comprising an in situ forming media configured to be introduced into the form and to form a protective barrier at least partially around a circumference of the first nerve end.
58. The system of alternative 51, further comprising an in situ forming media configured to be introduced into the form and to form a protective barrier at least partially around a circumference of the first nerve end and second nerve end.
59. The system of alternative 58, wherein the in situ forming media comprises a growth permissive gel configured to be introduced into a gap between the first nerve end and the second nerve end when the first nerve end and the second nerve end are positioned on the scaffold within the cavity of the form.
60. The system of alternative 57, wherein the in situ forming media comprises a non-growth permissive gel configured to at least partially integrate into the scaffold.
61. The system of alternative 57, wherein the in situ forming media is biodegradable.
62. The system of claim 61, wherein the in situ forming media biodegrades in about 3 months to about 3 years.
63. The system of alternative 62, wherein the in situ forming media biodegrades in about 3 months to about 6 months.
64. The system of alternative 50, wherein the scaffold comprises a technical textile.
65. The system of alternative 50, wherein the scaffold comprises a knitted material.
66. The system of alternative 65, wherein the scaffold comprises a warp knitted material.
67. The system of alternative 50, wherein the scaffold comprises a woven material.
68. The system of alternative 50, wherein the scaffold comprises a natural monofilament, polydioxanone, poly(lactic-co-glycolic acid), poliglecaprone, polyglactin, polyglycolic acid, polyglycolide fiber, polylactide fiber, collagen, alginate, chitosan, cellulose, or carboxymethylcellulose.
69. The system of alternative 68, wherein the scaffold comprises polyglycolide fiber
70. The system of alternative 69, wherein the scaffold comprises warp knitted polyglycolide fiber.
71. The system of alternative 50, wherein the scaffold is hydrophilic.
72. The system of alternative 50, wherein the scaffold has a thickness of less than or equal to about 1 mm.
73. The system of alternative 50, wherein the scaffold has a thickness of about 0.8 mm to about 1.2 mm.
74. The system of alternative 50, wherein the scaffold has a thickness of about 1 mm.
75. The system of alternative 50, wherein the scaffold has a coefficient of friction of about 0.8 to 1.
76. The system of alternative 50, wherein the scaffold has an elastic modulus of at least about 1 kPa.
77. The system of alternative 76, wherein the scaffold has an elastic modulus of about 15 kPa to about 45 kPa.
78. The system of alternative 50, wherein the scaffold has a tensile strength of at least about 10 N.
79. The system of alternative 78, wherein the scaffold has a tensile strength of about 70 N to about 100 N.
80. The system of alternative 50, wherein the scaffold has a plurality of pores.
81. The system of alternative 80, wherein the plurality of pores of the scaffold are each about 0.8 mm2 to about 1.2 mm2 in cross-sectional area.
82. The system of alternative 50, wherein the scaffold is biodegradable.
83. The system of alternative 82, wherein the scaffold biodegrades in about 1 month to about 6 months.
84. The system of alternative 83, wherein the scaffold biodegrades in about 6 weeks to about 8 weeks.

References in the specification to “one embodiment,” “an embodiment,” “an illustrative embodiment,” “some embodiments,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

As used in the description and claims, the singular form “a”, “an” and “the” include both singular and plural references unless the context clearly dictates otherwise. For example, the term “scaffold” may include, and is contemplated to include, a plurality of scaffolds. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of facilitating nerve growth, the method comprising:
positioning a scaffold in at least a portion of a cavity defined by a form;
positioning a first nerve end of a first nerve at least partially onto the scaffold in the cavity;
introducing an in situ forming media in contact with the first nerve end and the scaffold; and
permitting the in situ forming media to undergo a transformation from a first, relatively flowable state to a second, relatively non-flowable state to form a protective barrier surrounding the first nerve end.

2. The method of claim 1, wherein the method further comprises, positioning a second nerve end of a second nerve at least partially onto the scaffold in the cavity, wherein the in situ forming media is further in contact with the second nerve end, and wherein the in situ forming media further forms a protective barrier surrounding the first nerve end and the second nerve end.

3. The method of claim 2, wherein the introducing an in situ forming media comprises introducing a growth permissive gel and a non-growth permissive gel.

4. The method of claim 3, wherein the growth permissive gel is introduced into a gap between the first nerve end and the second nerve end.

5. The method of claim 3, wherein the growth permissive gel does not integrate into the scaffold and wherein the non-growth permissive gel integrates into the scaffold.

6. The method of claim 3, wherein the growth permissive gel comprises hyaluronic acid and water.

7. The method of claim 3, wherein the non-growth permissive gel comprises polyethylene glycol and water.

8. The method of claim 1, wherein the scaffold has a plurality of pores.

9. The method of claim 1, wherein the scaffold comprises a natural monofilament, polydioxanone, poly(lactic-co-glycolic acid), poliglecaprone, polyglactin, polyglycolic acid, polyglycolide fiber, polylactide fiber, collagen, alginate, chitosan, cellulose, or carboxymethylcellulose.

10. The method of claim 9, wherein the scaffold comprises polyglycolide fiber.

11. The method of claim 2, wherein the scaffold is configured to produce friction on the first nerve end and the second nerve end effective to hold the first nerve end and the second nerve end in a static position during the introducing of the in situ forming media.

12. A system for creating a protective barrier in situ around a nerve end or nerve to nerve junction, the system comprising:
a form comprising:
a sidewall defining a cavity and a top opening for accessing the cavity, the top opening configured to receive a first nerve end; and
a scaffold at least partially positioned on a bottom interior surface of the sidewall of the form such that the first nerve end is offset from the bottom interior surface of the sidewall when the first nerve end is positioned on the scaffold within the cavity.

13. The system of claim 12, wherein the top opening is configured to receive a second nerve end, and wherein the second nerve end is offset from the bottom interior surface of the sidewall when the second nerve end is positioned on the scaffold within the cavity.

14. The system of claim 12, wherein the form further comprises:
a first nerve guide comprising a first surface at least partially defining a first side access through the sidewall for positioning a first nerve end in the cavity, the first surface being on a first end of the form at a first elevated position relative to a bottom interior surface of the sidewall; and
a second nerve guide comprising a second surface at least partially defining a second side access through the sidewall for positioning a second nerve end in the cavity, the second surface being on a second end of the form at a second elevated position relative to a bottom interior surface of the sidewall.

15. The system of claim 13, further comprising an in situ forming media configured to be introduced into the form and to form a protective barrier at least partially around a circumference of the first nerve end and second nerve end.

16. The system of claim 15, wherein the in situ forming media comprises a growth permissive gel configured to be introduced into a gap between the first nerve end and the second nerve end when the first nerve end and the second nerve end are positioned on the scaffold within the cavity of the form.

17. The system of claim 12, wherein the scaffold comprises a natural monofilament, polydioxanone, poly(lactic-co-glycolic acid), poliglecaprone, polyglactin, polyglycolic acid, polyglycolide fiber, polylactide fiber, collagen, alginate, chitosan, cellulose, or carboxymethylcellulose.

18. The system of claim 17, wherein the scaffold comprises polyglycolide fiber.

19. The system of claim 12, wherein the scaffold has a thickness of about 0.8 mm to about 1.2 mm.

20. The system of claim 12, wherein the scaffold has a plurality of pores.
